(19) United States
(12) Patent Application Publication
(10) Pub. No.: US 2014/0179741 A1
(43) Pub. Date: Jun. 26, 2014

(54) USE OF CHELATORS OF DIVALENT CATIONS TO PROMOTE NERVE REGENERATION

(71) Applicants: MASSACHUSETTS INSTITUTE OF TECHNOLOGY, Cambridge, MA (US); CHILDREN'S MEDICAL CENTER CORPORATION, Boston, MA (US)

(72) Inventors: Larry I. Benowitz, Newton, MA (US); Stephen J. Lippard, Cambridge, MA (US); Paul Rosenberg, Newton, MA (US); Yiqing Li, Malden, MA (US); Zhen Huang, Arlington, MA (US)

(73) Assignees: MASSACHUSETTS INSTITUTE OF TECHNOLOGY, Cambridge, MA (US); CHILDREN'S MEDICAL CENTER CORPORATION, Boston, MA (US)

(21) Appl. No.: 14/050,657

(22) Filed: Oct. 10, 2013

Related U.S. Application Data
(60) Provisional application No. 61/712,074, filed on Oct. 10, 2012.

(51) Int. Cl. A61K 31/444; A61K 31/197

(52) U.S. Cl. CPC A61K 31/444; A61K 31/197
USPC 514/332; 514/566; 435/375

(57) ABSTRACT

Disclosed herein are methods to promote axonal outgrowth of a neuron comprising, contacting the neuron with an effective amount of a chelating agent, to thereby promote axonal outgrowth in the neuron. Also disclosed are methods of treating a subject for a CNS lesion, comprising, administering to the subject a therapeutically effective amount of a chelating agent, Also disclosed are devices for promoting regeneration in a lesioned neuron, and pharmaceutical compositions comprising a therapeutically effective amount of a chelating agent formulated for localized administration directly to an injured neuron. Examples of such chelating agents are provided.

### CROSS REFERENCE TO RELATED APPLICATIONS

This application claims benefit under 35 U.S.C. §119(e) of U.S. Provisional Application No. 61/712,074, filed Oct. 10, 2012, the content of which is incorporated fully herein by reference in its entirety.

### GOVERNMENTAL SUPPORT

This invention was made with Government support under DM102446 Contract W81XWH-11-2-0023 awarded by the U.S. Department of Defense, and GM065519 awarded by the National Institute of Health. The Government has certain rights in the invention.

### FIELD OF THE INVENTION

The present invention relates to the field of neurobiology and treatment of neurological disease and injury.

### BACKGROUND OF THE INVENTION

Under normal circumstances, neurons within the central nervous system (CNS) are unable to regenerate injured nerve fibers (axons), a condition that can result in irreversible losses of sensory, motor, autonomic, and/or cognitive functions depending on the site of damage. One widely studied example of a CNS pathway that normally cannot regenerate when injured is the optic nerve. The optic nerve injury serves as a useful model system for other CNS injuries. Following traumatic nerve injury, ischemic damage, or degenerative diseases such as glaucoma, the projection neurons of the eye, the retinal ganglion cells (RGCs), cannot regrow their axons and soon begin to die leaving victims with lifelong visual losses. Research has discovered ways to activate RGCs' intrinsic growth capacity and counteract extracellular signals that normally inhibit axon growth. Application of these methods has enabled the production of unprecedented levels of optic nerve regeneration in animal model systems. However, often these methods do not fully arrest the slow loss of RGCs that persists after axonal injury, and even the cells that partially regenerate their axons are likely to be in a compromised state. The identification of cellular and molecular mechanisms that cause RGCs to die after axotomy and establishment of ways to counteract these mechanisms, when combined with methods to promote axon regeneration, will restore meaningful levels of neuron regeneration and lost function.

### SUMMARY

One aspect of the invention relates to a method of promoting axonal outgrowth of a neuron. The method comprises contacting the neuron with an effective amount of a chelating agent, to thereby promote axonal outgrowth in the neuron. In one embodiment, the neuron is an injured neuron. In one embodiment, the injured neuron results from acute traumatic injury. In one embodiment of the methods described herein, the neuron is further contacted with one or more additional agents that promote axonal outgrowth. In one embodiment of the methods described herein the agent that promotes axonal outgrowth is selected from the group consisting of inosine, oncomodulin, a pten inhibitor, and combinations thereof. In one embodiment of the methods described herein the neuron is further contacted with an agent that increases cAMP. In one embodiment of the methods described herein the contacting occurs within a time frame following injury of the neuron selected from the group consisting of 12 hours, 24 hours, 36 hours, and 48 hours. In one embodiment of the methods described herein the contacting occurs within a time frame following injury of the neuron consisting of 1 day, 2 days, 3 days, 4 days, 5 days, 6 days, and 7 days. In one embodiment of the methods described herein the chelating agent binds zinc. In one embodiment of the methods described herein the chelating agent binds divalent cations intracellularly, extracellularly, or both intracellularly and extracellularly. In one embodiment of the methods described herein the chelating agent is selected from the group consisting of TPEN, ZX1, TPA, ZX1E, EDTA saturated with Ca²⁺, and combinations thereof.

Another aspect of the invention relates to a method of treating a subject for a CNS lesion. The method comprises administering to the subject a therapeutically effective amount of a chelating agent, wherein administering results in contacting one or more lesioned CNS neurons of the subject with the chelating agent, to thereby promote regeneration in the CNS neurons. In one embodiment, the subject is a human. In one embodiment of the herein described methods, the chelating agent is selected from the group consisting of TPEN, ZX1, TPA, ZX1E, EDTA saturated with Ca²⁺, and combinations thereof. In one embodiment of the herein described methods, the CNS lesion results from an acute traumatic injury. In one embodiment of the herein described methods, the acute traumatic injury is selected from the group consisting of crush, severing, and acute ischemia. In one embodiment of the herein described methods, administration first occurs prior to the injury. In one embodiment of the herein described methods, administration first occurs following the injury. In one embodiment of the herein described methods, administration is prior to the injury, and continues following the injury. In one embodiment of the herein described methods, administration first occurs following injury, within 12 hours, 24 hours, 36 hours, or 48 hours of the injury. In one embodiment of the herein described methods, administration results in continuous delivery for a period of 1 day, 2 days, 3 days, 4 days, 5 days, 6 days, or 7 days.

In one embodiment of the herein described methods, the CNS lesion results from an acute traumatic injury. In one embodiment of the herein described methods, the CNS lesion results from a traumatic brain injury. In one embodiment of the herein described methods, the CNS lesion results from a stroke. In one embodiment of the herein described methods, the lesioned CNS neuron is in the optic nerve. In one embodiment of the herein described methods, the CNS lesion results from an acute spinal cord injury. In one embodiment of the herein described methods, the lesioned CNS neuron is in the spinal cord of a patient, and the inhibitor is intrathecally administered to the patient. In one embodiment of the herein described methods, the lesioned CNS neuron is a sensory neuron.

In one embodiment of the herein described methods, the chelating agent is contacted to the neuron by administration via method selected from the group consisting of direct injection, intrathecally, ocularly, subdurally, extradurally, epidurally, and intramedullary. In one embodiment of the herein described methods, the chelating agent is contacted to the neuron by administration locally at the lesioned CNS neuron. In one embodiment of the herein described methods, the chelating agent is contacted to the neuron by administration locally at the site of axonal injury, or to the site of origin of the injured neuron. In one embodiment of the herein described methods, one or more additional agents that promote axonal outgrowth are administered to the subject or otherwise contacted to the injured neuron. In one embodiment of the herein described methods, the additional agent is selected from the group consisting of inosine, oncomodulin, an inhibitor of PTEN, and combinations thereof.

Another aspect of the invention relates to a device for promoting regeneration in a lesioned central nervous system (CNS) neuron. The device comprises a reservoir loaded with a premeasured and contained amount of a therapeutically effective amount of a chelating agent, or a composition described herein, and specifically adapted for implementing the methods described herein.

Another aspect of the invention relates to a pharmaceutical composition comprising a therapeutically effective amount of a chelating agent formulated for localized administration directly to an injured neuron. In one embodiment, the chelating agent is selected from the group consisting of TPEN, ZX1, TPA, ZX1E, EDTA saturated with Ca²⁺, and combinations thereof.

### BRIEF DESCRIPTION OF THE DRAWINGS

FIG. 1A-FIG. 1D shows results from experiments that indicate levels of free Zn²⁺ are elevated within 6 hours after optic nerve injury and are reduced with the chelator TPEN. A) and B) Mice underwent unilateral optic nerve injury and received i.p. injections of sodium selenite immediately afterwards. Levels of free Zn²⁺ increase within 6 hours. C) and D) TPEN (100 μM, 3 injections starting 1 day before nerve injury) suppresses the increase in free Zn²⁺. The temporal resolution of this method is limited by the time delay for TPEN to reach the retina.

FIG. 2 is a photograph that shows the elevation of free Zn²⁺ in the inner plexiform layer of the retina after optic nerve injury. Two distinct bands of free Zn²⁺ are visualized by autometallography (asterisks mark the relevant bands) within the inner plexiform layer (ipl). The ipl contains synaptic inputs from amacrine and horizontal cells upon the dendrites of RGCs. The cell bodies of RGCs lie within the ganglion cell layer (gcl) and are immunostained for βIII tubulin. Cell nuclei were also specifically visualized with DAPI. inl, inner nuclear layer; opl, outer plexiform layer; onl, outer nuclear layer.

FIG. 3 shows results of experiments that indicate chelating free Zn²⁺ promotes RGC survival. RGC survival was evaluated 2 weeks after optic nerve injury. In all cases, TPEN was injected intraocularly on the day of optic nerve injury and 4 days later; in the last 3 groups, it was also given one day before nerve injury. Mice in the last 2 groups had the pten gene excised in RGCs (see text). Survival is shown relative to normal mice. TPEN is maximally effective at 100 μM and there is no additional benefit from pre-treatment; the effect of pten gene deletion is not enhanced by TPEN (n=5-6 per group; treated groups all differ from untreated mice at P<0.01).

FIG. 4 shows results of experiments that indicate chelating free Zn²⁺ (with TPEN) stimulates optic nerve regeneration. Regenerating axons were quantified at 500 μm (left of each bar pair) and 1000 μm (right of each bar pair) beyond the injury site 2 weeks after optic nerve injury. TPEN was administered immediately after optic nerve injury and four days later in all cases; it was also administered one day beforehand in the last 3 groups. TPEN was maximally effective at 100-500 μM, and had a stronger effect when also administered beforehand. Zn²⁺ chelation strongly augmented the effect of pten gene deletion (n=5-6 for all groups; all P<0.01 compared to untreated mice).

FIG. 5 shows results from experiments that indicate extracellular Zn²⁺ suppresses RGC survival after optic nerve injury. ZX1 is a chelator of free Zn²⁺ that does not permeate the cell membrane, and thus only chelates extracellular Zn²⁺. When treatment begins prior to optic nerve surgery, ZX1 is equally protective against RGC death at all concentrations from 10 μM up (P<0.001). ZX1 is less protective when there is no pretreatment (compare ZX1 with and without pretreatment). ZX1 has about the same effect as PTEN, which chelates both intra- and extracellular Zn²⁺. All values are significantly higher than the negative control (optic nerve crush, no treatment: P<0.01).

FIG. 6A-FIG. 6C show results from experiments that indicate Chelation of free Zn²⁺ suppresses molecular changes that lead to RGC apoptosis. A) Cross-sections through the normal retina or 5 days after optic nerve crush, with or without treatment with the Zn²⁺ chelator TPEN. Sections were immunostained to detect the anti-apoptotic protein Bcl-xL or the pro-apoptotic enzymes Caspase-3 or -8. B) C) Quantitation of cellular changes (avg. number of stained cells per section). Optic nerve injury causes a marked elevation of Caspases-3 and -8. Whereas TPEN diminishes the number of Caspase-3 positive cells, it does not strongly affect levels of Caspase-8. BclxL levels have not been quantified yet, but appear to decrease with optic nerve injury and to be partially preserved by TPEN treatment. gcl: ganglion cell layer; ipl, inner plexiform layer; inl, inner nuclear layer.

FIG. 7 shows results from experiments that indicate Chelation of extracellular Zn²⁺ induces regeneration. TPEN, a membrane-permeable chelator of Zn²⁺, promotes axon regeneration after optic nerve injury. New results shown here indicate that ZX1, a chelator that cannot cross the cell membrane, is similarly effective in inducing regeneration. Therefore, the free Zn²⁺ that is responsible for inhibiting axon regeneration is extracellular. Note that ZX1 is maximally effective at 30 μM, above which its effects decrease. As in FIG. 1, ZX1 was delivered multiple times, including 1 day before optic nerve injury in the “Pre-treatment” group.

FIG. 8A-FIG. 8C shows results from experiments that indicate ZX1 promotes retinal ganglion cells (RGC) survival in adult mice. Retinas from different groups were stained and quantified at 2 weeks after optic nerve crush. A) B) Retinal whole mounts immunostained with antibodies to βIII-tubulin to visualize RGCs. A) Loss of RGCs at 2 weeks after optic nerve crush. B), Preservation of RGCs 2 weeks after optic nerve crush with 100 μM ZX1 intraocular injection. Scale bar, 50 μm. C) Quantitation of RGC survival after 2 weeks. *p<0.05, **p<0.01, ***p<0.001 compared with optic nerve crush alone. #p<0.05, compared with 10 μM ZX1 injection.

FIG. 9A-FIG. 9C shows results from experiments that indicate ZX1 enhances axon regeneration in the mouse optic nerve after injury. A) B) Longitudinal sections through the adult mouse optic nerve showing GAP-43-positive axons distal to the injury site (asterisks) 2 weeks after optic nerve crush. A) Absence of regeneration after crush alone. B) Increased regenerating after intraocular injection of ZX1 (100 μM). Scale bar, 100 μm. C) Quantitation of axon growth at indicated distances beyond the crush site. *p<0.05, **p<0.01, ***p<0.001 compared with optic nerve crush alone.

FIG. 10A-FIG. 10B shows results from experiments indicating the characterization of ZX1E (trappable ZX1). A) change of UV/vis spectrum of 3 in 25% MeOH/PBS upon addition of esterase at 37° C. The final spectrum matched the spectrum of ZX1 (B). A) Insert: the decrease of absorbance at 340 nm.

FIG. 11A-FIG. 11M show results from experiments indicating ionic Zn²⁺ increases in synaptic layers of the retina after optic nerve injury. (A-H) show ionic Zn²⁺ visualized in cross-sections of the mouse retina using autometallography (AMG). The AMG signal increases in the inner plexiform layer (IPL) of the retina within 6 h of optic nerve injury (B) and continues to increase at 24 h, particularly in specific sublaminae (C). At 3 da, the highest signal is seen within RGCs (D). (E) Quantitation of the AMG signal in the IPL. *P<0.05, ***P<0.001 compared to normal retina. (F) The AMG signal 6 h after nerve injury (pseudo-colored red) lies below the ganglion cell layer (GCL). RGCs are visualized by immunostaining for βIII tubulin (green). (G-H) Chelating Zn²⁺ reduces the AMG signal. (I-K). Detection of Zn²⁺ using the selective fluorescent sensor Zinpyr1 (ZP1). Increased Zinpyr1 staining is shown in the IPL 24 after optic nerve injury (cf. (I) and (J)), whereas at 3 d, highest levels are seen in RGCs (arrows). (I) ZnT3 (although shown in black and white, this was visualized optically by a red stain) is highly expressed in the IPL. The right side of the image shows double-labeling for ZnT3 and βIII tubulin, which stains RGCs and the overlying axons. Scale bar, 40 μm.

FIG. 12A-FIG. 12J shows results from experiments that indicate chelation of Zn²⁺ promotes RGC survival. (A). ZX1 augments RGC survival when injected intraocularly shortly after optic nerve crush (ONC) and 4 d later (***P<0.001). Pre-mixing ZX1 with Zn²⁺ but not Ca²⁺ diminishes this effect (if P<0.01). (B) Ca-EDTA, but not Zn-EDTA, promotes cell survival. (C-F) RGC survival, visualized in retinal whole-mounts stained for βIII tubulin, is increased by TPEN or ZX1. (G) Additional injection of either chelator 1 d before ONC has little benefit on RGC survival. Also, neither chelator augments survival after pten deletion. (H) Zn²⁺ chelation provides long-lasting neuroprotection. All RGCs die within 12 wk after ONC (solid lower line), but TPEN provides enduring protection (solid upper line). Deletion of pten initially provides strong protection, but this effect declines by 12 wks (dotted lower line). Addition of TPEN produces an enduring effect (dotted upper line). (I) TPEN is equally effective whether administered immediately after ONC or 3 d later, whereas the effectiveness of ZX1 declines administered at d 3 (††P<0.01).

FIG. 13A-FIG. 13G shows results from experiments that indicate chelation of Zn²⁺ stimulates axon regeneration. (A-C). Effect of Zn²⁺ chelators (delivered on days 0 and 4 after ONC) on regeneration (evaluated) at 2 weeks. Light shading: regenerating axons 500 μm distal to the injury site; dark shading: axons at 1 mm. (D-G). Regenerating axons in longitudinal sections of the optic nerve visualized by GAP-43 immunostaining. a, e. ZX1 (100 μM, 3 μl) promotes regeneration; this effect is eliminated by pre-mixing with equimolar Zn²⁺. (B). Ca-EDTA, but not Zn-EDTA, promotes regeneration. (C). (sets 1-4): pre-treatment with ZX1 or TPEN (additional injection one day before ONC) enhances the effects of injecting on D0 and D4 (*P<0.01 for TPEN) without enhancing cell survival (c.f. FIG. 12). ZX1 and TPEN double the effect of deleting the pten gene, especially for longer growth (G). This combination does not alter the effect of pten deletion on cell survival (FIG. 12). *P<0.05, **P<0.01, ***P<0.001 compared to single treatments.

FIG. 14A-FIG. 14H shows results from experiments that indicate delayed appearance of NO and possible relationship to Zn²⁺ in RGCs. (A-D): NO visualized using the fluorescent dye DAF-2/DA. A signal is detected beginning ˜3 days after optic nerve injury in the ganglion cell layer (arrows, C), and then appears throughout the inner plexiform layer (D). (E,F): Colocalization of NO (E) with βIII tubulin (F), a marker for RGCs. (G, H) Deletion of the gene for neuronal NO synthase (nNOS) increases RGC survival after optic nerve crush (G) and occludes the effect of TPEN(H). These results suggest that NO production and elevation of Zn²⁺ may be in the same pathway. G and H have different scales and slightly different baseline RGC survival due to differences in cell counting methods in the two data sets.

FIG. 15 shows experimental results the indicate chelating Zn²⁺ does not augment axon outgrowth in cultured RGCs. Adult rat RGCs were cultured in defined media (Yin 2003) in the absence or presence of forskolin (F, 10 μM), mannose (M, 100 μM), and oncomodulin (Ocm, 16 nM), without (left bar of each pair) or with (right bar of each pair) the Zn²⁺ chelator ZX1 (10 μM). As expected, F+M induced moderate outgrowth that was enhanced by Ocm. ZX1 had no effect, suggesting that its large effect in vivo (FIG. 13) may be non-cell autonomous.

FIG. 16 shows experimental results that indicate blockade of voltage-gated K+ channels promotes RGC survival and occludes the effect of Zn²⁺ chelation. Agitoxin (AgTx2, 50 μM), an inhibitor of Kv1 channels, slightly improves RGC survival after optic nerve injury (*P<0.05) but does not augment the effect of TPEN. TEA has a strong effect (**P<0.001) at 100 μM, a concentration that only blocks Kv's 1, 3 and 7.

FIG. 17 shows experimental results that indicate chelation of free Zn²⁺ suppresses changes associated with apoptosis. Retinal cross-sections from normal mice or 5 days after optic nerve crush, with or without TPEN treatment. Sections were immunostained for the anti-apoptotic protein Bcl-xL or the pro-apoptotic enzymes Caspase-3 or -8. Optic nerve injury causes a marked elevation of Caspases-3 and -8 and a decline in Bcl-xL. TPEN diminishes the expression of Caspases-3 and -8 and attenuates the decline in Bcl-xL gcl: ganglion cell layer; ipl, inner plexiform layer; inl, inner nuclear layer.

FIG. 18 shows results from experiments that indicate a zinc chelator elevates regeneration above previously reported methods. The effects on neuroregeneration of zinc chelator ZX1 combined with other known effectors (pten inhibition, oncomodulin promotion) was investigated. Pten inhibition was achieved by deletion of the pten gene, and oncomodulin increases was achieved by administration of Zymosan and elevation of cAMP levels. Combination of zinc chelation with either effector was seen to further enhance the neuronal regeneration observed in the absence of zinc chelation.

### DETAILED DESCRIPTION OF THE INVENTION

Aspects of the invention relate to the finding that divalent cations, such as zinc play an important role in death pathways in neurons in the central nervous system (CNS). The presence of divalent cations such as zinc inhibit neuronal survival and regrowth in a neuronal injury. The minimization of these divalent cations in the neuronal environment, e.g., with chelators, serves to promote regeneration of injured neurons. Without being bound by theory, it is thought that this occurs by blocking an early step in the death pathway activated following injury, and through a distinct mechanism activating, or permitting activation of, neurons' potential for axon growth. These findings can be applied therapeutically, alone and in combination with other approaches, to disorders and diseases of the CNS characterized by axonal injury such as spinal cord trauma, optic nerve injury, glaucoma, multiple sclerosis, stroke, neonatal brain injury, and CNS trauma. In one aspect, the therapeutic application of these findings will allow regeneration of the injured neurons.

### DEFINITIONS

As used herein, the term neuronal “growth” or “outgrowth” includes the process by which, axons or dendrites extend from a neuron. This is also referred to in the art as neurite outgrowth. The outgrowth can result in a new neuritic projection or in the extension of a previously existing cellular process. Neuronal outgrowth can be measured by the number of neurons extending neuritic projections or processes, or by the length of the linear extensions, or a combination of both. Neuronal growth processes, including neuritogenesis, can be evidenced by GAP-43 expression detected by methods such as immunostaining “Stimulating neuronal growth” means promoting neuronal outgrowth. Neurite outgrowth or neuritogenesis is meant to encompass outgrowth of a neuron which results in either an axon or a dendrite.

As used herein, the term “CNS neurons” is intended to include the neurons of the brain, eye, the cranial nerves and the spinal cord.

As used herein, the term “administering” to a patient includes dispensing, delivering or applying an active compound in a pharmaceutical formulation to a subject by any suitable route for delivery of the active compound to the desired location in the subject. Administration may be localized or systemic in a subject and includes delivery by either the parenteral or oral route, intramuscular injection, subcutaneous/intradermal injection, intravenous injection, buccal administration, transdermal delivery, use of nanoparticles, use of locally applied matrix containing therapeutic agent, and administration by the rectal, colonic, vaginal, intranasal or respiratory tract route. The agents may, for example, be administered to a comatose, anesthetized or paralyzed subject, or may be administered to a pregnant subject to stimulate axonal growth in a fetus, or in a neonate. Specific routes of administration may include topical application (such as by eyedrops, creams or erodible formulations to be placed under the eyelid), intraocular injection into the aqueous or the vitreous humor, injection into the external layers of the eye, such as via subconjunctival injection or subtenon injection, parenteral administration or via oral routes.

As used herein, the term “intrathecal administration” is intended to include delivering an inhibitor(s) formulation directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head.

The term “cerebral ventricle” is intended to include the cavities in the brain that are continuous with the central canal of the spinal cord. Administration of an agent to any of the above mentioned sites can be achieved by direct injection of the agent formulation or by the use of infusion pumps. For injection, the agent formulation of the invention can be formulated in liquid solutions, preferably in physiologically compatible buffers such as Hank's solution or Ringer's solution. In addition, the agent formulation may be formulated in solid form and re-14666420.2 dissolved or suspended immediately prior to use. Lyophilized forms are also included. The injection can be, for example, in the form of a bolus injection or continuous infusion (e.g., using infusion pumps) of the inhibitor(s) formulation.

As used herein, the term “contacting CNS neurons” refers to any mode of agent delivery or “administration” either to cells, or to whole organisms in which the agent is capable of exhibiting its pharmacological effect in neurons. “contacting CNS neurons” is intended to include both in vivo and in vitro methods of bringing an agent of the invention into proximity with a neuron. Suitable modes of administration can be determined by those skilled in the art and such modes of administration may vary between agents. For example, when axonal growth of CNS neurons is stimulated ex vivo, agents can be administered, for example, by transfection, lipofection, electroporation, viral vector infection, or by addition to growth medium.

As used herein, “effective amount” of an agent is an amount sufficient to achieve a desired therapeutic or pharmacological effect, such as an amount sufficient to remove a significant amount of metal ions from the neuronal environment, or an amount that is capable of promoting regeneration of CNS neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

A therapeutically effective amount or dosage of an agent is one that results in detectable therapeutic benefit to the individual. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an active compound can include a single treatment or a series of treatments. A therapeutically effective amount may range from about 0.001 to 30 mg/kg body weight, with other ranges of the invention including about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, and 5 to 6 mg/kg body weight. In one example, a subject is treated with an agent in the range of between about 0.1 to 20 mg/kg body weight, one time per week for between about 1 to 10 weeks, alternatively between 2 to 8 weeks, between about 3 to 7 weeks, or for about 4, 5, or 6 weeks. It will also be appreciated that the effective dosage of an agent used for treatment may increase or decrease over the course of a particular treatment. The agents of the present invention can be administered simultaneously or separately.

As used herein, the term “patient” or “subject” or “animal” or “host” refers to any mammal. The patient is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the term “neurological disorder” is intended to include a disease, disorder, or condition which directly or indirectly affects the normal functioning or anatomy of a subject's nervous system.

As used herein, the term “stroke” is art recognized and is intended to include sudden diminution or loss of consciousness, sensation, and voluntary motion caused by rupture or obstruction (for example, by a blood clot) of an artery of the brain.

As used herein, “traumatic brain injury” is art recognized and is intended to include the condition in which, a traumatic blow to the head causes damage to the brain or connecting spinal cord, often without penetrating the skull. Usually, the initial trauma can result in expanding hematoma, subarachnoid hemorrhage, cerebral edema, raised intracranial pressure, and cerebral hypoxia, which can, in turn, lead to severe secondary events due to low cerebral blood flow.

As used herein, the term “cAMP modulator” includes any compound which has the ability to modulate the amount, production, concentration, activity or stability of cAMP in a cell, or to modulate the pharmacological activity of cellular cAMP. cAMP modulators may act at the level of adenylate cyclase, upstream of adenylate cyclase, or downstream of adenylate cyclase, such as at the level of cAMP itself, in the signaling pathway that leads to the production of cAMP. Cyclic AMP modulators may act inside the cell, for example at the level of a G-protein such as Gi, Go, Gq, Gs and Gt, or outside the cell, such as at the level of an extra-cellular receptor such as a G-protein coupled receptor. Cyclic AMP modulators include activators of adenylate cyclase such as forskolin; nonhydrolyzable analogues of cAMP including 8-bromo-cAMP, 8-chloro-cAMP, or dibutyryl cAMP (db-cAMP); isoprotenol; vasoactive intestinal peptide; PACAP; calcium ionophores; membrane depolarization; phosphodiesterase inhibitors such as pentoxifylline and theophylline; specific phosphodiesterase IV (PDE IV) inhibitors; and beta 2-adrenoreceptor agonists such as salbutamol. The term cAMP modulator also includes compounds which inhibit cAMP production, function, activity or stability, such as phosphodiesterases, such as cyclic nucleotide phosphodiesterase 3B. cAMP modulators which inhibit cAMP production, function, activity or stability are known in the art and are described in, for example, in Nano et al., Pflugers Arch 439 (5): 547-54, 2000, the contents of which are incorporated herein by reference.

As used herein, the term “treating” includes reducing or alleviating at least one adverse effect or symptom of a condition, disease or disorder associated with a CNS lesion. Treating may result in the promotion of a significant amount of neuronal outgrowth in a subject (e.g., at the lesion site). Treating may result in the reduction of a symptom and/or a biochemical marker of such a condition (e.g., by at least 10%). As alternative examples, a detectable reduction in a symptom, for example, an increase in function, mobility, or sensation, (e.g., by 10%).

### DESCRIPTION

One aspect of the invention relates to a method of promoting axonal outgrowth of a neuron by contacting the neuron with an effective amount of a chelating agent (e.g., a zinc chelator), to thereby promote axonal outgrowth in the neuron. Examples of such chelating agents are described herein and otherwise known in the art. In one embodiment, the neuron is injured, such as results from a physical injury to a subject, or from a disease or disorder that causes injury. The neuron may also be contacted with one or more additional agents that promote axonal outgrowth. Examples of such agents are provided herein or otherwise known in the art. Contacting cells and/or cellular elements in the immediate vacinity to the neuron in a similar manner, may also produce a similar effect.

In one embodiment, the contacting of the chelating agent to the neuron (e.g., via administration to an injured subject) occurs within a recent time frame of the injury. Examples of such time frames, include, without limitation, contacting within 12 hours following the injury. Other such time frames are contacting the neuron within 24, 36, and 48 hours of the injury. Other such time frames are contacting the neuron within 1, 2, 3, 4, 5, 6, and 7 days of the injury. Contacting at a later point following the injury may also have some benefit. Contacting can be ongoing or repeated, following the initial contact. Method of promoting the required contact (e.g., in a subject) are described herein.

Another aspect of the invention relates to a method of treating a subject for a neuronal disorder. The method involves administering to the subject a therapeutically effective amount of a chelating agent. Administration results in promotion of neuronal outgrowth in the subject at a location which is expected to ameliorate symptom, alleviate discomfort, or lead to improvement of the condition of the subject with respect to the disorder. The administering typically will result in contacting one or more neurons of the subject (e.g., lesioned neurons) with the chelating agent, to thereby promote outgrowth (e.g., regeneration) of the neurons. Contacting cells and/or cellular elements in the immediate vacinity to the neuron in a similar manner, may also produce a similar effect. Examples of appropriate routes of administration are provided herein.

### Chelators of Divalent Cations

Chelators of divalent cations are envisioned as chelating agents for use in the methods and compositions described herein. Such chelators that have been characterized that preferentially chelate zinc, or that have high affinity for zinc in addition to one or more other ions, such as calcium or copper, are envisioned as chelating agents for use in the methods and compositions described herein. A “chelating agent,” as used herein, is a compound having sites (one, two, three, four or more) which can simultaneously bind to one or more divalent cations (e.g., a metal ion such as zinc, calcium, cobalt, iron, manganese, or copper ions, or other divalent ions such as lead, etc.). The binding sites typically comprise oxygen, nitrogen, sulfur or phosphorus. For example, salts of EDTA (ethylenediaminetetraacetic acid) can form at least four to six bonds with a metal ion or metal ions via the oxygen atoms of four acetic acid moieties and the nitrogen atoms of ethylenediamine moieties of EDTA. It is understood that a chelating agent also includes a polymer which has multiple binding sites to a metal or metal ions. Preferably, a chelating agent of the invention is non-toxic and does not cause unacceptable side effects at the dosages being administered. In one embodiment, the chelating agent is a zinc-chelating agent. A “zinc-chelating agent” refers to a chelating agent which can bind to a zinc ion or zinc ions. This may be in the context of also binding other ions. In one embodiment, the chelating agent is a zinc-specific chelating agent. A “zinc-specific chelating agent” refers to a chelating agent which binds a zinc ion or zinc ions preferentially to any other ions. The chelating agent may bind the divalent cation(s) intracellularly or extracellularly.

Examples of chelating agents (not all of which are membrane permeable) include, without limitation, ZX1 ((2-((Bis(pyridin-2-ylmethyl)amino)methylamino)benzenesulfonic acid; Pan, et al., Neuron 2011, 71, 1116-1126.), ZX1E (described herein in Example 3), TPA (Tris[(2-pyridyl)methyl]amine), phanquinone (4,7-phenanthroline-5,6-dione), clioquinol (PN Gerolymatos SA), chloroquinol, penicillamine, trientine, N,N′-diethyldithiocarbamate (DDC), 2,3,2′-tetraamine (2,3,2′-tet), neocuproine, N,N,N′,N′-tetrakis(2-pyridylmethyl) ethylenediamine (TPEN), 1,10-phenanthroline (PHE), tetraethylenepentamine (TEPA), triethylene tetraamine and tris(2-carboxyethyl)phosphine (TCEP), bathophenanthroline disulfonic acid (BPADA), Ethylenediaminetetraacetic acid (EDTA), ethylene glycol (bis) aminoethyl ether tetra acetic acid (EGTA), nitrilotriacetic acid, TIRON™, N,N-bis(2-hydroxyethyl)glycine (bicine); O,O′-bis(2-aminophenyl ethylene glycol) ethylenediamine-N,N,N′,N′-tetraacetic acid (BAPTA), trans-1,2-diamino cyclohexane-ethylenediamine-N,N,N′,N′-tetraacetic acid (CyDTA), 1,3-diamino-2-hydroxy-propane-ethylenediamine-N,N,N′, N′-tetraacetic acid (DPTA-OH), ethylene-diamine-N,N′-dipropionic acid dihydrochloride (EDDP), ethylenediamine-N,N′-bis(methylenephosphonic acid) hemihydrate (EDDPO), ethylenediamine-N,N,N′,N′-tetrakis(methylenephosphonic acid) (EDTPO), N,N′-bis(2-hydroxybenzyl)ethylene diamine-N,N′-diacetic acid (HBED), 1,6-hexamethylenediamine-N,N,N′,N′-tetraacetic acid (HDTA, or HEDTA), N-(2-hydroxyethyl)iminodiacetic acid (HIDA), iminodiacetic acid (IDA), 1,2-diaminopropane-N,N,N′,N′-tetraacetic acid (methyl-EDTA), nitriltriacetic acid (NTA), nitrilotripropionic acid (NTP), nitrilotris (methylenephosphonic acid) trisodium salt (NTPO), triethylenetetramine-N,N,N′,N″,N″-hexaacetic acid (TTHA), bathocuproine, bathophenanthroline, TETA, citric acid, salicylic acid, and malic acid, and analogues and derivatives, including hydrophobic derivatives and pharmaceutically acceptable salts thereof. A combination of two or more chelating agents may also be used. In one embodiment, one or more of the specific chelators is specifically excluded from the methods and compositions disclosed herein. In one embodiment of the methods described herein are performed in the absence of one or more of the specific chelators disclosed herein (e.g., in the absence of one or more of EDTA, EGTA, clinoquol, or in the absence of a heterocyclic compound having two fused 6-membered rings with nitrogen atoms at positions 1 and 3, a carboxy group at position 4, and a hydroxy group and position 8, with both rings being aromatic (U.S. Pat. No. 8,084,459, the contents of which are specifically incorporated herein by reference). In one embodiment, the compositions herein lack one or more specific chelators disclosed herein (e.g., one or more of EDTA, EGTA, clinoquol or the heterocyclic compound discussed directly above (U.S. Pat. No. 8,084,459)).

Suitable membrane-permeable chelating agents include, without limitation TPEN; 1,10-O-phenanthroline; and diethyldithiocarbamate (DEDC), TPA, and ZX1E. Persons of skill in the art will be able to determine suitable compounds by routine testing using the methods described herein and known in the art.

In one embodiment, EDTA or EGTA, pre-saturated with a cation such as Ca²⁺ or Mg²⁺, is envisioned. In one embodiment, the cation is not Zn²⁺, but rather is a cation for which EDTA or EGTA has a lower affinity than for Zn²⁺. Without being bound by theory, it is thought that the presence of the cation increases the permeability of the EDTA or EGTA. Once the composition enters the tissue, Zn²⁺ is exchanged for the pre-saturated cation. In one embodiment, the pre-saturated EDTA or EGTA is administered to a subject in a site specific manner, rather than systemically.

Agents that Activate the Growth Pathway of CNS Neurons

The method described herein can be performed in combination with (e.g., with co-administration of) a second agent that promotes axonal growth, otherwise activates the growth pathway of a neuron, or otherwise promotes a neurosalutary effects. As used herein, a “neurosalutary effect” means a response or result favorable to the health or function of a neuron, of a part of the nervous system, or of the nervous system generally. Examples of such effects include improvements in the ability of a neuron or portion of the nervous system to resist insult, to regenerate, to maintain desirable function, to grow or to survive. The phrase “producing a neurosalutary effect” includes producing or effecting such a response or improvement in function or resilience within a component of the nervous system. For example, examples of producing a neurosalutary effect would include stimulating neuronal outgrowth after injury to a neuron; rendering a neuron resistant to apoptosis; rendering a neuron resistant to a toxic compound such as β-amyloid, ammonia, or other neurotoxins; reversing age-related neuronal atrophy or loss of function; or reversing age-related loss of cholinergic innervation.

Some preferred agents include but are not limited to inosine, mannose, gulose, or glucose-6-phosphate, as described in Li et. al., 2003, J. Neuroscience 23(21):7830-7838; Chen Et al., 2002, Proc. Natl. Acad. Sci. U.S.A, 99:1931-1936; and Benowitz et al., 1998 J. Biol. Chem. 273:29626-29634, which are herein incorporated by reference in their entirety. TGF-β, and oncomodulin as described in Yin et al., 2003, J. Neurosci., 23: 2284-2293, are also preferred agents. Other agents are PTEN inhibitors (U.S. Patent Application Publication 2009/0305333) and SOCS3 inhibitors (U.S. Patent Application Publication 2011/0124706). In addition, polypeptide growth factors such as BDNF, NGF, NT-3, CNTF, LIF, and GDNF can be used. In one embodiment the methods of the present invention further comprise contacting CNS neurons with a cAMP modulator that increases the concentration of intracellular cAMP. For example, the ability of mature rat retinal ganglionic cells to respond to mannose requires elevated cAMP (Li et. al., 2003, J. Neuroscience 23(21):7830-7838).

The ability of an agent to produce neuronal outgrowth of CNS neurons in a subject may be assessed using any of a variety of known procedures and assays. For example, the ability of an agent to re-establish neural connectivity and/or function after a CNS injury, may be determined histologically (either by slicing neuronal tissue and looking at neuronal branching, or by showing cytoplasmic transport of dyes). Agents may also be assessed by monitoring the ability of the agent to fully or partially restore the electroretinogram after damage to the neural retina or optic nerve; or to fully or partially restore a pupillary response to light in the damaged eye.

Other tests that may be used to determine the ability of an agent to produce neuronal outgrowth in a subject include standard tests of neurological function in human subjects or in animal models of spinal injury (such as standard reflex testing, urologic tests, urodynamic testing, tests for deep and superficial pain appreciation, propnoceptive placing of the hind limbs, ambulation, and evoked potential testing). In addition, nerve impulse conduction can be measured in a subject, such as by measuring conduct action potentials, as an indication of the production of a neurosalutary effect.

Animal models suitable for use in the assays of the present invention include the rat model of partial transaction (described in Weidner et al., (2001) Proc Natl Acad Sci USA 98:3513-3518). This animal model tests how well a compound can enhance the survival and sprouting of the intact remaining fragment of an almost fully-transected cord. Accordingly, after administration of a candidate agent these animals may be evaluated for recovery of a certain function, such as how well the rats may manipulate food pellets with their forearms (to which the relevant cord had been cut 97%).

Another animal model suitable for use in the assays of the present invention includes the rat model of stroke as described by Kawamata et al., ((1997) Proc Natl Acad Sci USA 94:8179-8184), which describes in detail various tests that may be used to assess sensor motor function in the limbs as well as vestibulomotor function after an injury. Administration to these animals of the agents described herein can be used to assess whether a given compound, route of administration, or dosage results in neuronal outgrowth or a neurosalutary effects, such as increasing the level of function, or increasing the rate of regaining function or the degree of retention of function in the test animals.

Standard neurological evaluations used to assess progress in human patients after a stroke may also be used to evaluate the ability of an agent to produce a neurosalutary effect in a subject. Such standard neurological evaluations are routine in the medical arts, and are described in, for example, “Guide to Clinical Neurobiology” Edited by Mohr and Gautier (Churchill Livingstone Inc. 1995).

### Pharmaceutically Acceptable Formulations

The therapeutic agent, or combination of agents, described herein can be contained in pharmaceutically acceptable formulations, otherwise referred to herein as a pharmaceutical composition. Such a pharmaceutically acceptable formulation may include a pharmaceutically acceptable carrier(s) and/or excipient(s). As used herein, “pharmaceutically acceptable carrier” includes any and all solvents, dispersion media, coatings, antibacterial and anti fungal agents, isotonic and absorption delaying agents, and the like that are physiologically compatible. For example, the carrier can be suitable for injection into the cerebrospinal fluid. Excipients include pharmaceutically acceptable stabilizers. The present invention pertains to any pharmaceutically acceptable formulations, including synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes. In one embodiment, the pharmaceutical composition is formulated for localized administration directly to an injured neuron (e.g., at the site of origin of the injured neuron or at the site of axonal injury).

In one embodiment, the pharmaceutically acceptable formulations comprise a polymeric matrix. The terms “polymer” or “polymeric” are art-recognized and include a structural framework comprised of repeating monomer units which is capable of delivering an agent that promotes axonal outgrowth such that treatment of a targeted condition, such as a neurological disorder, occurs. The terms also include co-polymers and homopolymers such as synthetic or naturally occurring. Linear polymers, branched polymers, and cross-linked polymers are also meant to be included.

For example, polymeric materials suitable for forming the pharmaceutically acceptable formulation employed in the present invention, include naturally derived polymers such as albumin, alginate, cellulose derivatives, collagen, fibrin, gelatin, and polysaccharides, as well as synthetic polymers such as polyesters (PLA, PLGA), polyethylene glycol, poloxomers, polyanhydrides, cyclodextrins, and pluronics. These polymers are biocompatible with the nervous system, including the central nervous system, they are biodegradable within the central nervous system without producing any toxic byproducts of degradation, and they possess the ability to modify the manner and duration of the active compound release by manipulating the polymer's kinetic characteristics. As used herein, the term “biodegradable” means that the polymer will degrade over time by the action of enzymes, by hydrolytic action and/or by other similar mechanisms in the body of the subject. As used herein, the term “biocompatible” means that the polymer is compatible with a living tissue or a living organism by not being toxic or injurious and by not causing an immunological rejection. Polymers can be prepared using methods known in the art.

The polymeric formulations can be formed by dispersion of the active compound within liquefied polymer, as described in U.S. Pat. No. 4,883,666, the teachings of which are incorporated herein by reference or by such methods as bulk polymerization, interfacial polymerization, solution polymerization and ring polymerization as described in Odian G., Principles of Polymerization and ring opening polymerization, 2nd ed., John Wiley & Sons, New York, 1981, the contents of which are incorporated herein by reference. The properties and characteristics of the formulations are controlled by varying such parameters as the reaction temperature, concentrations of polymer and the active compound, the types of solvent used, and reaction times.

The active therapeutic compound can be encapsulated in one or more pharmaceutically acceptable polymers, to form a microcapsule, microsphere, or microparticle, terms used herein interchangeably. Microcapsules, microspheres, and microparticles are conventionally free-flowing powders consisting of spherical particles of 2 millimeters or less in diameter, usually 500 microns or less in diameter. Particles less than 1 micron are conventionally referred to as nanocapsules, nanoparticles or nanospheres. For the most part, the difference between a microcapsule and a nanocapsule, a microsphere and a nanosphere, or microparticle and nanoparticle is size; generally there is little, if any, difference between the internal structure of the two. In one aspect of the present invention, the mean average diameter is less than about 45 μm, preferably less than 20 μm, and more preferably between about 0.1 and 10 μm.

In another embodiment, the pharmaceutically acceptable formulations comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used.

Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing an active compound such variables as the efficiency of active compound encapsulation, labiality of the active compound, homogeneity and size of the resulting population of vesicles, active compound-to-lipid ratio, permeability, instability of the preparation, and pharmaceutical acceptability of the formulation should be considered.

Prior to introduction, the formulations can be sterilized, by any of the numerous available techniques of the art, such as with gamma radiation or electron beam sterilization.

### Administration of the Pharmaceutically Acceptable Formulations to a Subject

Administration is to a subject by a route that results in contacting an effective amount of one or more of the therapeutic agents described herein to the target neuron(s). In one embodiment, administration of the therapeutic agent to a subject (e.g., in a single or in different pharmaceutical compositions, with or without an additional factor described herein) results in the therapeutic agent directly contacting an injured neuron in need of regeneration (e.g., at the site of axonal injury or at the site of origin of the injured neuron). In one embodiment, administration results in contacting neurons proximal to a site of neuronal injury. In one embodiment, the administration is directly to an injured neuron (e.g., at the site of origin of the injured neuron or at the site of axonal injury). Such administration can be achieved by localized or systemic administration.

The term “administering” to a subject includes dispensing, delivering or applying an active compound in a pharmaceutical formulation to a subject by any suitable route for delivery of the active compound to the desired location in the subject, (e.g., the injury, the injured neuron, or the site of desired outgrowth of the neuron). This includes, without limitation, delivery by either the parenteral or oral route, intramuscular injection, subcutaneous/intradermal injection, intravenous injection, buccal administration, transdermal delivery and administration by the rectal, colonic, vaginal, intranasal or respiratory tract route, intraocular, ocular. Another form of administration suitable for treatment of spinal cord injury is injection into the spinal column or spinal canal.

Specific routes of administration and the dosage regimen will be determined by skilled clinicians based on factors such as the exact nature of the condition being treated, the severity of the condition, and the age and general physical condition of the patient.

Administration to the subject can be by any one or combination of a variety of methods (e.g., parenterally, enterally and/or topically). The appropriate method(s) will depend upon the circumstances of the individual (e.g. the location of the target neuron(s), the condition of the individual, the desired duration of the contact, whether local or systemic treatment is desired). The administration can be by any methods described herein that will result in contact of sufficient therapeutic agent(s) to the target neuron to promote survival and/or regeneration.

Since regeneration and axonal generation in the treatment of a neuronal injury includes compensatory promotion of neuronal outgrowth of uninjured neurons, benefit is expected from mere delivery of the agent to an injury site. As such, suitable target neurons are actual damaged neurons, and also neurons that are in the immediate area of an injury site. The specific location and extent of an injury site can be determined by the skilled practioner. Examples of injury sites are the site of physical damage or disruption of neuronal activity. The immediate area of an injury site will vary with respect to the specific injury, the nature of the injury, and the nature of the injured neurons (e.g., axonal length, specific function, etc.) and can be determined by the skilled practitioner. In one embodiment, the immediate area of the injury site is within about 1-10 mm of identified damaged neurons (e.g., 1, 2, 3, 4, 5, 6, 7, 8, 9, or 10 mm).

In one embodiment, the administration is to the lesioned neuron(s), but is to a location that is not at the injury site.

In one embodiment, the administration is localized so as to be highly targeted to a specific site. In one embodiment, the administration is systemic, and results in delivery of the appropriate concentration to the specific site.

When the agents are delivered to a patient, they can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally.

Both local and systemic administration are contemplated by the invention. Desirable features of local administration include achieving effective local concentrations of the active compound as well as avoiding adverse side effects from systemic administration of the active compound. In one embodiment, the therapeutic agents are administered by introduction into the cerebrospinal fluid of the subject. In certain aspects of the invention, the therapeutic agent is introduced into a cerebral ventricle, the lumbar area, or the cisterna magna. In another aspect, the therapeutic agent is introduced locally, such as into the site of nerve or cord injury, into a site of pain or neural degeneration, or intraocularly to contact neuroretinal cells.

The pharmaceutically acceptable formulations can be suspended in aqueous vehicles and introduced through conventional hypodermic needles or using infusion pumps.

In one embodiment, the therapeutic agent formulation described herein is administered to the subject in the period from the time of, for example, an injury to the CNS up to about 100 hours after the injury has occurred, for example within 24, 12, or 6 hours from the time of injury.

In one embodiment, the therapeutic agent formulation is administered into a subject intrathecally. As used herein, the term “intrathecal administration” is intended to include delivering an active compound formulation directly into the cerebrospinal fluid of a subject, by techniques including lateral cerebroventricular injection through a burrhole or cisternal or lumbar puncture or the like (described in Lazorthes et al., 1991, and Ommaya A. K., 1984, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The ten-n “cerebral ventricle” is intended to include the cavities in the brain that are continuous with the central canal of the spinal cord. Administration of a therapeutic agent to any of the above mentioned sites can be achieved by direct injection of the active compound formulation or by the use of infusion pumps. Implantable or external pumps and catheter may be used.

For injection, the active agent can be formulated in liquid solutions, preferably in physiologically compatible buffers such as Hank's solution or Ringer's solution or saline. In addition, the agent be formulated in solid form and re-dissolved or suspended immediately prior to use. Lyophilized forms are also included. The injection can be, for example, in the form of a bolus injection or continuous infusion (such as using infusion pumps) of the agent formulation.

In one embodiment of the invention, the formulation is administered by lateral cerebroventricular injection into the brain of a subject, preferably within 100 hours of when an injury (resulting in a condition characterized by aberrant axonal outgrowth of central nervous system neurons) occurs (such as within 6, 12, or 24 hours of the time of the injury). The injection can be made, for example, through a burr hole made in the subject's skull. In another embodiment, the formulation is administered through a surgically inserted shunt into the cerebral ventricle of a subject, preferably within 100 hours of when an injury occurs (such as within 6, 12 or 24 hours of the time of the injury). For example, the injection can be made into the lateral ventricles, which are larger, even though injection into the third and fourth smaller ventricles can also be made. In yet another embodiment, the formulation is administered by injection into the cisterna magna, or lumbar area of a subject, preferably within 100 hours of when an injury occurs (such as within 6, 12, or 24 hours of the time of the injury).

An additional means of administration to intracranial tissue involves application of compounds of the invention to the olfactory epithelium, with subsequent transmission to the olfactory bulb and transport to more proximal portions of the brain. Such administration can be by nebulized or aerosolized preparations.

In another embodiment, the formulation is administered to a subject at the site of injury, preferably within 100 hours of when an injury occurs (such as within 6, 12, or 24 hours of the time of the injury).

In a further embodiment, formulations for ophthalmic administration are used to prevent or reduce damage to retinal and optic nerve head tissues, as well as to enhance functional recovery after damage to ocular tissues. Ophthalmic conditions that may be treated include, but are not limited to, retinopathies (including diabetic retinopathy and retrolental fibroplasia), macular degeneration, ocular ischemia, glaucoma. Other conditions to be treated with the methods of the invention include damage associated with injuries to ophthalmic tissues, such as ischemia reperfusion injuries, photochemical injuries, and injuries associated with ocular surgery, particularly injuries to the retina or optic nerve head by exposure to light or surgical instruments. The ophthalmic formulation may also be used as an adjunct to ophthalmic surgery, such as by vitreal or subconjunctival injection following ophthalmic surgery. The formulation may be used for acute treatment of temporary conditions, or may be administered chronically, especially in the case of degenerative disease. The ophthalmic formulation may also be used prophylactically, especially prior to ocular surgery or noninvasive ophthalmic procedures or other types of surgery.

In one embodiment, the therapeutic agents described herein are contacted with the neuron using an implantable device that contains the therapeutic agent and that is specifically adapted for delivery to a neuron. Examples of devices include solid or semi-solid devices such as controlled release biodegradable matrices, fibers, pumps, stents, adsorbable gelatin (e.g. Gelfoam), etc. The device may be loaded with premeasured, discrete and contained amounts of the agents sufficient to promote regeneration and/or survival of the neuron. In one embodiment, the device provides continuous contact of the neuron with the agent at nanomolar or micromolar concentrations, (e.g., for at least 2, 5, or 10 days, or for at least 2, 3, or 4 weeks, or for greater than 4 weeks, e.g., 5, 6, 7, or 8 weeks).

In one embodiment, the agent is contacted in vivo by introduction into the central nervous system of a subject, e.g., into the cerebrospinal fluid of the subject. In certain aspects of the invention, the agent is introduced intrathecally, e.g., into a cerebral ventricle, the lumbar area, or the cisterna magna. In another aspect, the agent is introduced intraocullarly, to thereby contact retinal ganglion cells or the optic nerve. Modes of administration are described in U.S. Pat. No. 7,238,529.

In one embodiment of the invention, the therapeutic agent is administered by lateral cerebro ventricular injection into the brain of a subject in the inclusive period from the time of the injury to a time determined by the skilled practitioner (e.g., 100 hours). The injection can be made, for example, through a burr hole made in the subject's skull. In another embodiment, said encapsulated therapeutic agent is administered through a surgically inserted shunt into the cerebral ventricle of a subject in the inclusive period from the time of the injury to a time determined by the skilled practitioner (e.g., 100 hours thereafter). For example, the injection can be made into the lateral ventricles, which are larger, even though injection into the third and fourth smaller ventricles can also be made.

In yet another embodiment, the therapeutic agent is administered by injection into the cisterna magna, or lumbar area of a subject in the inclusive period from the time of the injury to a time determined by the skilled practitioner (e.g., 100 hours thereafter). Administration can be continuous, or can be by repeated doses.

In one embodiment, the repeated doses are formulated so that an effective amount of the therapeutic agent is continually present at the injury site.

In one embodiment, administration occurs following neuronal injury in the subject, not prior to or at the time of neuronal injury.

### Duration and Levels of Administration

Depending on the intended route of delivery, the therapeutic formulations may be administered in one or more dosage form(s) (e.g. liquid, ointment, solution, suspension, emulsion, tablet, capsule, caplet, lozenge, powder, granules, cachets, douche, suppository, cream, mist, eye drops, gel, inhalant, patch, implant, injectable, infusion, etc.). The dosage forms may include a variety of other ingredients, including binders, solvents, bulking agents, plasticizers etc.

In one embodiment, the therapeutic composition is administered to a subject for an extended period of time to produce optimum neuronal outgrowth. Sustained contact with the active compound can be achieved by, for example, repeated administration of the active compound over a period of time, such as one week, several weeks, one month or longer. More preferably, the formulation used to administer the active compound provides sustained delivery, such as “slow release” of the active compound to a subject. For example, the formulation may deliver the active compound for at least one, two, three, or four weeks after the formulation is administered to the subject. Preferably, a subject to be treated in accordance with the present invention is treated with the formulation for at least 30 days (either by repeated administration or by use of a sustained delivery system, or both).

As used herein, the term “sustained delivery” is intended to include continual delivery of the therapeutic agent in vivo over a period of time following administration, preferably at least several days, a week, several weeks, one month or longer. Sustained delivery of the therapeutic agent can be demonstrated by, for example, the continued therapeutic effect of the active compound over time (such as sustained delivery of the agents can be demonstrated by continued axonal growth in CNS neurons in a subject). Alternatively, sustained delivery of the therapeutic agent may be demonstrated by detecting the presence of the active agent in vivo over time.

Preferred approaches for sustained delivery include use of a polymeric capsule, a minipump to deliver the formulation, a biodegradable implant. Implantable infusion pump systems (such as Infusaid) and osmotic pumps (sold by Alza Corporation) are available in the art. Another mode of administration is via an implantable, externally programmable infusion pump. Suitable infusion pump systems and reservoir systems are also described in U.S. Pat. No. 5,368,562 by Blomquist and U.S. Pat. No. 4,731,058 by Doan, developed by Pharmacia Deltec Inc.

It is to be noted that dosage values may vary with the severity of the condition to be alleviated. It is to be further understood that for any particular subject, specific dosage regimens should be adjusted over time according to the individual need and the professional judgment of the person administering or supervising the administration of the agent and that dosage ranges set forth herein are exemplary only and are not intended to limit the scope or practice of the claimed invention.

The amount of agent administered to the individual will depend on the characteristics of the individual, such as general health, age, sex, body weight and tolerance to drugs as well as the degree, severity and type of rejection. The skilled artisan will be able to determine appropriate dosages depending on these and other factors. Typically, an effective amount can range from about 0.1 mg per day to about 100 mg per day for an adult. Preferably, the dosage ranges from about 1 mg per day to about 100 mg per day.

### Delivery Device

Another aspect of the invention relates to a device for promoting regeneration in a lesioned neuron. The device may be implantable into the subject. The device may have a reservoir loaded with a premeasured and contained amount of the therapeutic formulation. The device may be specifically adapted for delivery to a region of the body having one or more lesionsed CNS neurons. In one embodiment, the device is specifically adapted for delivery to a neuron. Examples of devices include solid or semi-solid devices such as controlled release biodegradable matrices, fibers, pumps, stents, adsorbable gelatin (e.g. Gelfoam), etc. The device may be loaded with premeasured, discrete and contained amounts of the therapeutic agent sufficient to promote regeneration and/or survival of the neuron. In one embodiment, the device provides continuous contact of the neuron with the agent at nanomolar or micromolar concentrations, (e.g., for at least 2, 5, or 10 days, or for at least 2, 3, or 4 weeks, or for greater than 4 weeks, e.g., 5, 6, 7, or 8 weeks).

### Detection of Effects

Survival of a neuron is indicated by the number of neurons surviving from a specific injury or condition, as compared to the number of neurons surviving as a result of the effects of the administered agent (e.g., zinc chelator), and also by the length of time the survival persists, as compared to the length of time survival persists as a result of the effects of the administered agent. Survival is considered to be significant if it persists for an extended period of time post-injury (e.g., greater than 2 weeks post-injury, greater than 3 weeks, and greater than 4 weeks post-injury). In one embodiment, greater than 10% of neurons (e.g., 15%, 20%, 25%, 30%, 35%, 0%, 45%, 50%, 55%, 60%, 65%, 70% and 75%), survive for an extended period of time post-injury. In one embodiment, greater than 20% of neurons survive for an extended period of time post-injury.

Regeneration is indicated by the number of neurons (injured and also uninjured) and by extended length of the axonal outgrowth of the neurons, as compared to the number of neurons and extended length of the axonal outgrowth of the neurons that results from the effects of the adminstered agent, and by the time frame post-injury that the outgrowth occurs, as compared to the time frame post-injury that outgrowth occurs resulting from the effects of the administered agent. Regeneration and axonal outgrowth occurs if greater than 10% or greater than 20% (e.g., 15%, 20%, 25%, 30%, 35%, 0%, 45%, 50%, 55%, 60%, 65%, 70% and 75%) of the neurons regenerate injured axons or generate new axons, that extend at least 0.5 mm distal to the lesion epicenter. In one embodiment, greater than 10% or greater than 20% (e.g., 15%, 20%, 25%, 30%, 35%, 0%, 45%, 50%, 55%, 60%, 65%, 70% and 75%) of neurons regenerate injured axons or generate axons over 1 mm distal to the lesion site. In one embodiment, greater than 10% (e.g., 15%, 20%, 25%, 30%, 35%, 0%, 45%, 50%, 55%, 60%, 65%, 70% and 75%) or greater than 20% of neurons regenerate or generate new axons that extend at least 2 mm distal from the lesion site.

Regeneration and neuronal outgrowth is also indicated by a significant amount of outgrowth having occurred on or after 2 weeks post-injury. For example significant outgrowth occurs for up to 3 weeks or 4 weeks post-injury. Regeneration and neuronal outgrowth can also be indicated by restoration of function to the neuron. Function of a neuron can be detected by a variety of methods known in the art.

### Neurons

The methods and compositions described herein are suited for the promotion of survival, neuronal regeneration and axonal outgrowth of CNS (central nervous system) neurons. In one embodiment the neuron is a terminally differentiated neuron. In one embodiment, the neuron is an adult neuron (e.g, in a subject that has reached maturity, such as in humans older than 18 years). In one embodiment, the neuron is non-embryonic. In one embodiment, the neuron is in an immature organism (e.g., embryo, infant, child).

All CNS neurons are suitable for such methods described herein. CNS neurons include, without limitation, a cerebellar granule neuron, spinal cord neuron, or an ocular neuron. In one embodiment, the neuron is the optic nerve. In one embodiment, the neuron is a sensory neuron (e.g., dorsal root ganglion (DRG) sensory neuron). In one embodiment, the CNS neuron is known or determined to be under specific regeneration inhibition. Such determination can be determined by the skilled practitioner.

### Neuronal Lesions

As used in the art, the term lesion refers to damage (e.g., to a system or a cell). Damage to a system is evidenced by aberrant function, reduction of function, loss of function of the system, or loss of essential components (e.g., specialized cells such as neurons). Damage to a specific neuron is also evidenced by aberrant function, loss of function, reduced function, and/or cell death. Some forms of damage to a neuron can be directly detected (e.g., by visualization as with a severed or crushed neuronal axon). Neuronal lesions can result from a variety of insults, including, injury, toxic effects, atrophy (e.g., due to lack of trophic factors). Injuries that typically cause neuronal lesions include, without limitation, severing and crushing. A neuronal lesion, as the term is used herein, results from damage to the neuron. Such damage may be complete loss of a neuron, or loss of a part of the neuron (e.g., an axon). Such damage may results from acute or traumatic injury to the neuron (e.g., crush, severing) such as the result of external trauma to the subject (e.g., contusion, laceration, acute spinal cord injury, traumatic brain injury, cortical impact, etc.). Acute traumatic injury to a neuron can also result from an acute condition, such as stroke, that results in acute ischemia to the neuron resulting in acute damage. The specific location of neuronal damage will vary with the specific cause of the damage, and the specific individual. In one embodiment of the invention described herein, the lesioned CNS neuron is located in CNS white matter, particularly white matter that has been subjected to traumatic injury.

Damage to a neuron may also be incurred from a chronic injury (e.g., repetitive stress injury) or condition (e.g., chronic inflammation or disease). Chronic injury leads to neurodegeneration such as caused by neurotoxicity or a neurological disease or disorder (e.g. Huntington's disease, Parkinson's disease, Alzheimer's disease, multiple system atrophy (MSA), etc.). In one embodiment, the damage is not incurred from a chronic neurodegenerative disease, such as Alzheimer's disease.

In one embodiment of the invention, damage results from an ocular injury or disorder (e.g. toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, eye damage from free radicals, etc.), or an optic neuropathy (e.g. ischemic optic neuropathies, toxic optic neuropathies, ocular ischemic syndrome, optic nerve inflammation, infection of the optic nerve, optic neuritis, optic neuropathy, papilledema, papillitis, retrobulbar neuritis, commotio retinae, glaucoma, macular degeneration, retinitis pigmentosa, retinal detachment, retinal tears or holes, diabetic retinopathy, iatrogenic retinopathy, optic nerve drusen, etc.).

Damage to a neuron can be detected by the skilled practitioner through a variety of assays known in the art. Loss of function assays can be used to determine neuronal damage. Physical damage to the neuron (e.g., axonal crushing or severing) can sometimes be observed diagnostically through routine methods. One way to detect a lesion is through detection of axotomy-induced stress and/or pathology-induced down-regulation of protein translation (e.g., detected directly, indirectly, or inferred).

Such lesions may results from an injury to the nerve. An injury can be ongoing (e.g., the result of a disease or toxin) or acute (e.g., a traumatic injury). Such injuries may be caused by a physical external trauma experienced by a subject (e.g., resulting in neuronal crush or severing) or caused by an internal injury (e.g., which results in acute ischemia) such as a stroke, anneurism. Examples of such injuries include, without limitation, traumatic brain injury, spinal cord injury, stroke, optic nerve injury, toxic injuries, injuries to cranial nerves, and cerebral aneurism. Neurological lesions associated with ophthalmic conditions can also be treated with the methods described herein. Such injuries include, without limitation, retina and optic nerve damage, glaucoma and age related macular degeneration.

### Treatment of Neurological Disorders

Elements of the nervous system subject to disorders which may be effectively treated with the compounds and methods of the invention include the central, somatic, autonomic, sympathetic and parasympathetic components of the nervous system, neurosensory tissues within the eye, ear, nose, mouth or other organs, as well as glial tissues associated with neuronal cells and structures. Neurological disorders may be caused by an injury to a neuron, such as a mechanical injury or an injury due to a toxic compound, by the abnormal growth or development of a neuron, or by the misregulation, such as downregulation, of an activity of a neuron. Neurological disorders can detrimentally affect nervous system functions such as the sensory function (the ability to sense changes within the body and the outside environment); the integrative function (the ability to interpret the changes); and the motor function (the ability to respond to the interpretation by initiating an action such as a muscular contraction or glandular secretion).

Examples of neurological disorders include traumatic (e.g., acute) or toxic injuries to cranial nerves, spinal cord or to the brain, cranial nerves, traumatic brain injury, stroke, cerebral aneurism, and spinal cord injury. Other neurological disorders include cognitive and neurodegenerative disorders such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, hereditary motor and sensory neuropathy (Charcot-Marie-Tooth disease), diabetic neuropathy, progressive supranuclear palsy, epilepsy, and Jakob-Creutzfieldt disease. Autonomic function disorders include hypertension and sleep disorders.

As used herein, the term “acute” is used in reference to the timing of an injury. An acute injury is one which has taken place within a few days and is not ongoing.

### In Vitro Treatment of Neurons

Neurons derived from the central or peripheral nervous system can be contacted with the agents ex vivo to modulate axonal outgrowth in vitro. Accordingly, neurons can be isolated from a subject and grown in vitro, using techniques well known in the art, and then treated in accordance with the present invention to modulate axonal outgrowth. Briefly, a neuronal culture can be obtained by allowing neurons to migrate out of fragments of neural tissue adhering to a suitable substrate (such as a culture dish) or by disaggregating the tissue, such as mechanically or enzymatically, to produce a suspension of neurons. For example, the enzymes trypsin, collagenase, elastase, hyaluronidase, DNase, pronase, dispase, or various combinations thereof can be used. Methods for isolating neuronal tissue and the disaggregation of tissue to obtain isolated cells are described in Freshney, Culture of Animal Cells, A Manual of Basic Technique, Third Ed., 1994, the contents of which are incorporated herein by reference.

Such cells can be subsequently contacted with the agents (alone or in combination with a cAMP modulator) in amounts and for a duration of time as described above. Once modulation of axonal outgrowth has been achieved in the neurons, these cells can be re-administered to the subject, such as by implantation.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.

1. A method of promoting axonal outgrowth of a neuron comprising contacting the neuron with an effective amount of a chelating agent, to thereby promote axonal outgrowth in the neuron.
2. The method of paragraph 1, wherein the neuron is an injured neuron.
3. The method of paragraph 2, wherein the injured neuron results from acute traumatic injury.
4. The method of any one of paragraphs 1-3, wherein the neuron is further contacted with one or more additional agents that promote axonal outgrowth.
5. The method of paragraph 4, wherein the agent that promotes axonal outgrowth is selected from the group consisting of inosine, oncomodulin, a pten inhibitor, and combinations thereof.
6. The method of paragraph 4, wherein the neuron is further contacted with an agent that increases cAMP.
7. The method of any one of paragraphs 1-6, wherein the contacting occurs within a time frame following injury of the neuron selected from the group consisting of 12 hours, 24 hours, 36 hours, and 48 hours.
8. The method of any one of paragraphs 1-6, wherein the contacting occurs within a time frame following injury of the neuron consisting of 1 day, 2 days, 3 days, 4 days, 5 days, 6 days, and 7 days.
9. The method of any one of paragraphs 1-8, wherein the chelating agent binds zinc.
10. The method of any one of paragraphs 1-8, wherein the chelating agent binds divalent cations intracellularly, extracellularly, or both intracellularly and extracellularly.
11. The method of any one of paragraphs 1-8, wherein the chelating agent is selected from the group consisting of TPEN, ZX1, TPA, ZX1E, EDTA saturated with Ca²⁺, and combinations thereof.
12. A method of treating a subject for a CNS lesion, comprising, administering to the subject a therapeutically effective amount of a chelating agent, wherein administering results in contacting one or more lesioned CNS neurons of the subject with the chelating agent, to thereby promote regeneration in the CNS neurons.
13. The method of paragraph 12, wherein the subject is a human.
14. The method of any one of paragraphs 12-13, wherein the chelating agent is selected from the group consisting of TPEN, ZX1, TPA, ZX1E, EDTA saturated with Ca²⁺, and combinations thereof.
15. The method of any one of paragraphs 12-14, wherein the CNS lesion results from an acute traumatic injury.
16. The method of paragraph 15, wherein the acute traumatic injury is selected from the group consisting of crush, severing, and acute ischemia.
17. The method of any one of paragraphs 12-16 wherein administration first occurs prior to the injury.

18. The method of any one of paragraphs 12-16 wherein administration first occurs following the injury.

19. The method of paragraph 18 wherein administration first occurs within 12 hours, 24 hours, 36 hours, or 48 hours of the injury.
20. The method of any one of paragraphs 12-16, wherein administration results in continuous delivery for a period of 1 day, 2 days, 3 days, 4 days, 5 days, 6 days, or 7 days.
21. The method of paragraph 12, wherein the CNS lesion results from an acute traumatic injury.
22. The method of paragraph 12 wherein the CNS lesion results from a traumatic brain injury.
23. The method of paragraph 12 wherein the CNS lesion results from a stroke.
24. The method of paragraph 12 wherein the lesioned CNS neuron is in the optic nerve.
25. The method of paragraph 12 wherein the CNS lesion results from an acute spinal cord injury.
26. The method of paragraph 12 wherein the lesioned CNS neuron is in the spinal cord of a patient, and the inhibitor is intrathecally administered to the patient.
27. The method of paragraph 12 wherein lesioned CNS neuron is a sensory neuron.
28. The method of any one of paragraphs 12-27 wherein the chelating agent is administered by a method selected from the group consisting of direct injection, intrathecally, ocularly, subdurally, extradurally, epidurally, and intramedullary.
29. The method of any one of paragraphs 12-27 wherein the chelating agent is administered locally at the lesioned CNS neuron.
30. The method of paragraph 29, wherein the chelating agent is administered locally at the site of axonal injury, or to the site of origin of the injured neuron.
31. The method of any one of paragraphs 12-30, wherein one or more additional agents that promote axonal outgrowth are administered to the subject.
32. The method of paragraph 31, wherein the additional agent is selected from the group consisting of inosine, oncomodulin, an inhibitor of PTEN, and combinations thereof
33. A device for promoting regeneration in a lesioned central nervous system (CNS) neuron, comprising a reservoir loaded with a premeasured and contained amount of a therapeutically effective amount of a chelating agent, and specifically adapted for implementing the method of paragraph 12.
34. A pharmaceutical composition comprising a therapeutically effective amount of a chelating agent formulated for localized administration directly to an injured neuron.
35. The pharmaceutical composition of paragraph 34, wherein the chelating agent is selected from the group consisting of TPEN, ZX1, TPA, ZX1E, EDTA saturated with Ca²⁺, and combinations thereof.

The invention is further illustrated by the following examples, which should not be construed as further limiting.

### EXAMPLES

### Example 1

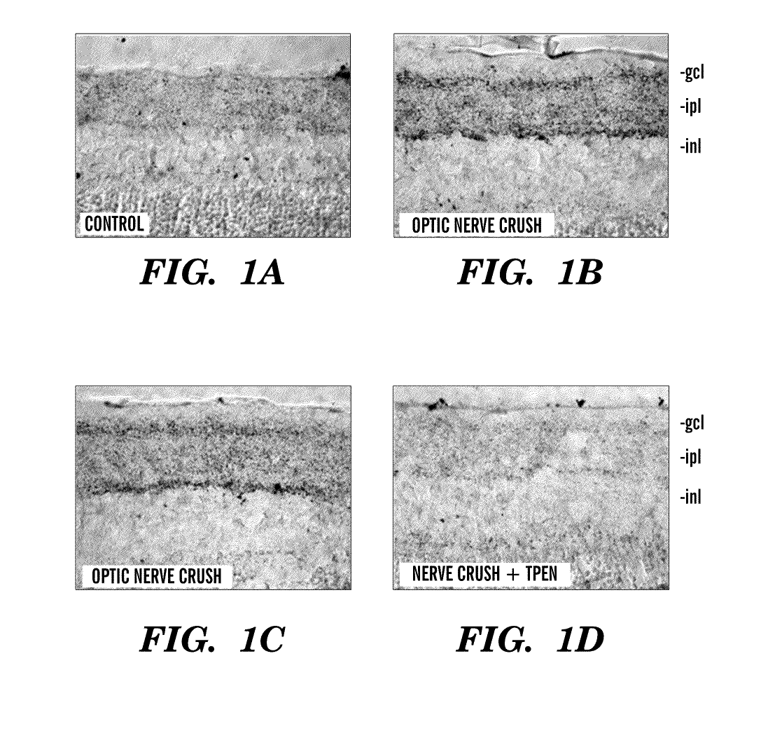

We investigated the hypothesis that the liberation of free Zn2+ plays a role in RGC death after axonal injury. We used zinc-selenium autometallography (ZnSeAMG) to investigate the rise in free Zn2+ after optic nerve injury, and found a marked elevation within 6 hours (FIG. 1. We also showed that TPEN, a chelator of free Zn2+, diminishes Zn2+ levels if injected prior to and after optic nerve injury (FIG. 1). Injection after injury also produces decrease.

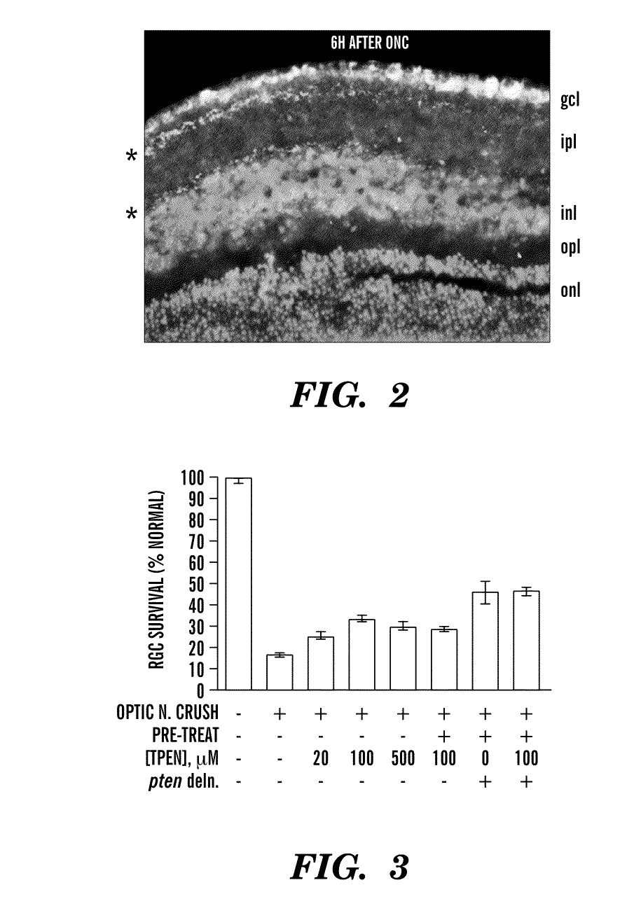

We have begun to investigate the cellular localization of free Zn2+ to understand how and where free Zn2+ leads to RGC death. We combined autometallography to visualize free Zn2+ with immunostaining with antibodies to βIII tubulin to visualize RGCs and DAPI staining to visualize all cell nuclei. As shown in FIG. 2, free Zn2+ accumulates in the inner plexiform layer (ipl) of the retina, which contains the synaptic inputs from amacrine cells and bipolar cells onto the dendrites of RGCs. This pattern suggests that synapses that arise from particular amacrine cells or bipolar cells could be the source of free Zn2+. Microglia may also be a source.

We carried out a dose-response study to determine the most effective concentration and treatment regimen with TPEN. As shown in FIG. 3, TPEN was most effective when delivered intraocularly at 100 μM (day 0 and day 4 after optic nerve crush). The effect of TPEN on RGC survival was not augmented by pre-administration of TPEN one day prior to optic nerve crush (FIG. 3, group 6). However, none of the treatments fully protected RGCs.

We also tested whether Zn²⁺ chelation can augment the effect of deleting the gene for PTEN (phosphatase and tensin homolog). PTEN suppresses signaling through the PI3 kinase pathway, and deletion of the pten gene increases RGC survival and axon regeneration after optic nerve injury (Park K K, et al. (2008) Science 322:963-966). We deleted the pten gene in RGCs by injecting adeno-associated virus expressing Cre recombinase (AAV2-Cre) into the eyes of mice having a conditional deletion of the pten gene. The tropism of AAV2 for RGCs causes the gene to be deleted selectively in RGCs via Cre-lox recombination. pten deletion had a strong effect on RGC survival, as expected (Park K K, et al. (2008) Science 322:963-966), and the addition of TPEN had no additional effect (FIG. 3, last 2 groups).

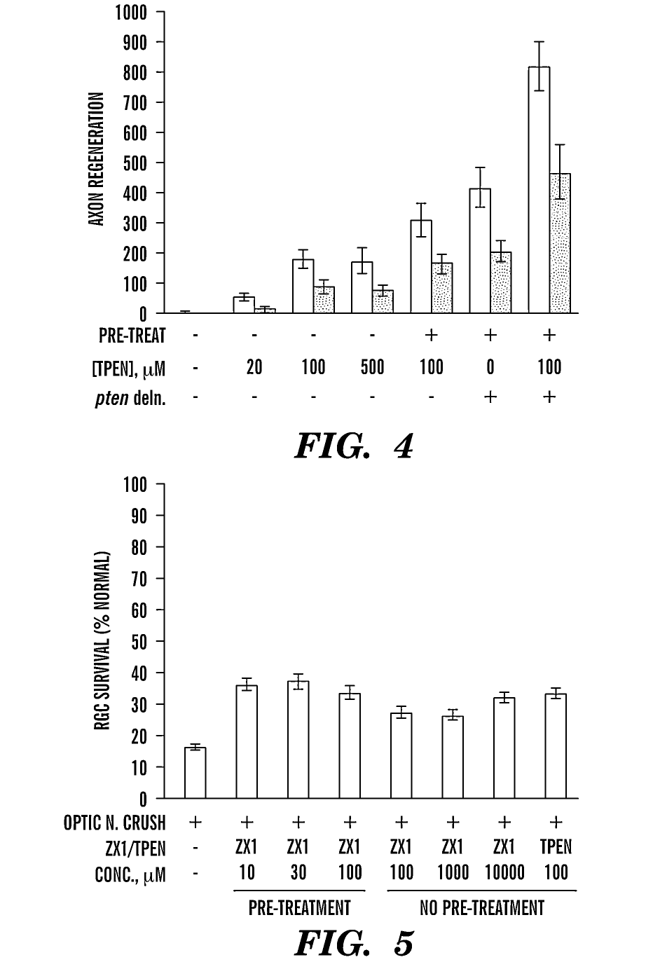

We have further investigated the effects of chelating free Zn²⁺ on axon regeneration. Previous experiments cited above indicated that Zn²⁺ chelation promotes optic nerve regeneration. We have carried out more extensive studies along these lines, establishing the optimal concentration of TPEN to stimulate regeneration and examining the effect of combining Zn²⁺ chelation with pten gene deletion. Chelating free Zn²⁺ (immediately after optic nerve crush and 4 days later) promoted regeneration, with a maximal effect achieved with 100 μM TPEN (FIG. 4: groups 3 and 4 compared to group 1). We also investigated whether the effect of free Zn²⁺ in suppressing regeneration occurs very rapidly, in which case TPEN introduced at the time of injury might not reach the appropriate sites in the retina soon enough to reverse the deleterious effects of free Zn²⁺. To test this possibility, we investigated whether pre-treatment with TPEN would have a stronger effect than post-surgical treatment alone. Remarkably, although pretreatment with TPEN failed to increase RGC survival (FIG. 1), it markedly increased the number of regenerating axons compared with post-operative treatment alone (FIG. 4, Group 5). Finally, we have found that chelating free Zn²⁺ augments the effects of a complementary proregenerative treatment. As shown in FIG. 4, chelating free Zn²⁺ (100 μM TPEN) and deleting the pten gene in RGCs had much greater effects on regeneration than either one alone, despite the fact that the two treatments did not have additive effects on RGC survival (FIG. 3). These results indicate that (1) the effect of chelating free Zn²⁺ on regeneration is separate from the effects of chelating Zn²⁺ on RGC survival; and (2) chelating free Zn²⁺ and deleting the pten gene have independent and additive effects on regeneration (but not on RGC survival). Combining Zn²⁺ chelation with pten inhibition is expected to be valuable clinically for improving nerve regeneration such as the optic nerve.

We next investigated whether the liberation of free Zn²⁺ plays a role in RGC death after axonal injury. As noted above, zinc-selenium autometallography (ZnSeAMG) demonstrated a marked rise in free Zn²⁺ in the retina within 6 hours after optic nerve injury, and TPEN, a chelator of free Zn²⁺, partially suppressed the loss of RGCs. The rise in free Zn²⁺ occurred selectively in two sub-layers of the inner plexiform layer (ipl) of the retina. The ipl contains synaptic inputs from amacrine cells and bipolar cells onto the dendrites of RGCs. These sub-layers may correspond to the synaptic inputs from either horizontal cells or specific types of amacrine cells onto the dendrites of particular types of RGCs.

We continued to investigate how free Zn²⁺ contributes to RGC death by examining whether its actions are intra- or extracellular. In the studies discussed above, we used TPEN to chelate free Zn²⁺. Because TPEN is membrane-permeable, we cannot determine whether its effects on cell survival are via chelation of intra- or extracellular Zn²⁺. To distinguish between these possibilities, we used ZX1, a Zn²⁺ chelator that cannot cross the cell membrane. As shown in FIG. 5, ZX1 was just as effective as TPEN in attenuating RGC death. Together, these results indicated that the free Zn²⁺ that is released following axonal injury is extracellular and is concentrated in particular synaptic layers of the retina. These results are surprising in view of the fact that the RGCs are the only cell directly affected by injury to the optic nerve, yet the primary change we observed was occurring at synapses on these cells' dendrites.

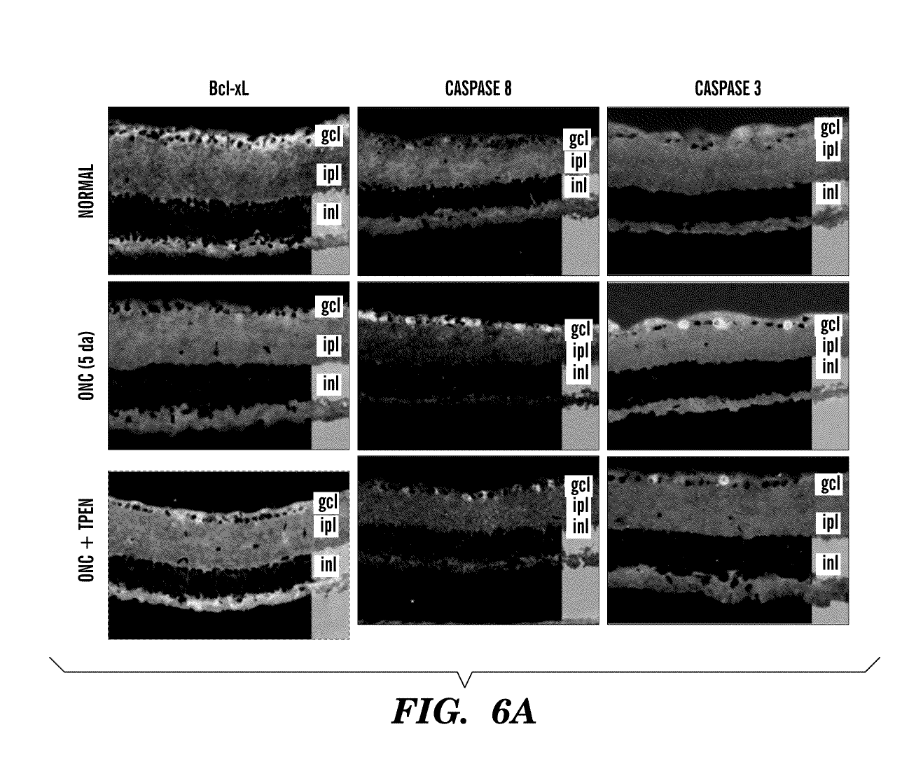

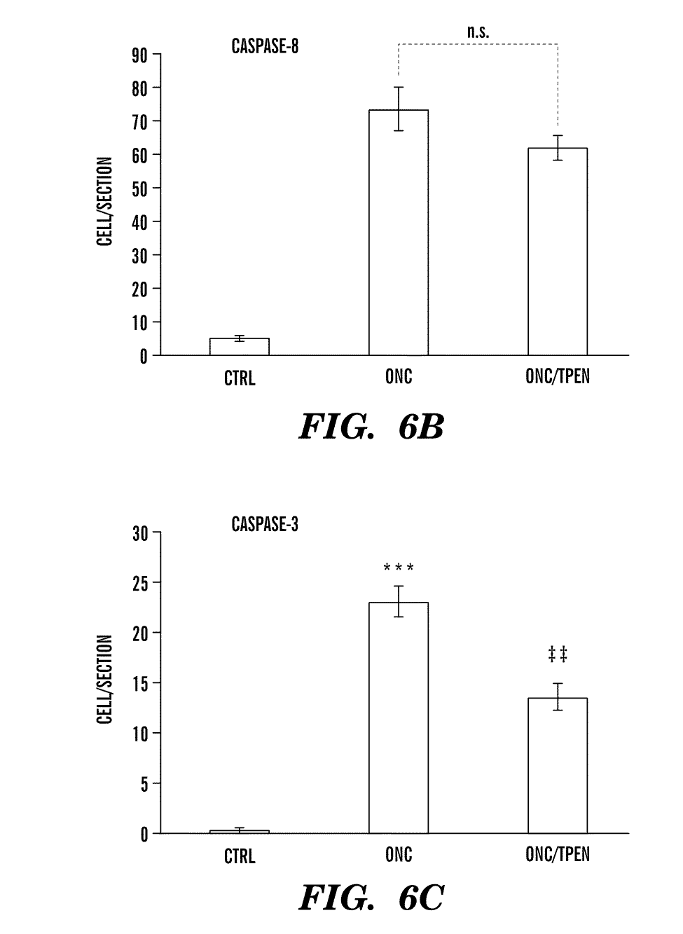

We have also studied the downstream pathways that are affected by blocking free Zn²⁺. Other groups have shown that RGC death is associated with diminished levels of the anti-apoptotic protein bcl-xL (Isenmann et al., (2003) Prog Retin Eye Res 22:483-543) and with increased levels of activated Caspase-3 (Kermer et al., (1999) FEBS Lett 453:361-364). As shown in FIG. 6, the Zn²⁺ chelater TPEN suppressed both the decrease in bcl-xL and the increase in Caspase-3. These results show that elevation of free Zn²⁺ is a major part of the pathway that leads to apoptotic death of RGCs after axonal injury, and that blocking the increase in free Zn²⁺ suppresses the apoptotic death of RGCs.

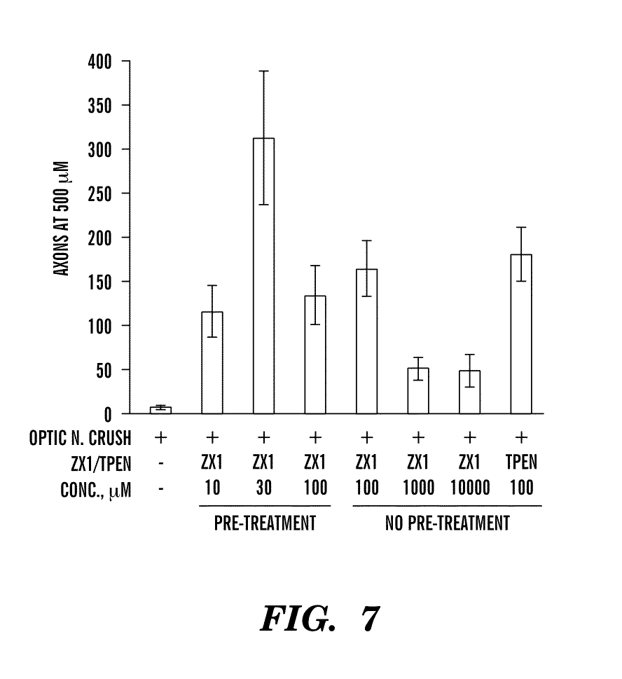

In addition ZX1, the chelator of extracellular Zn²⁺ mentioned above, also induces extensive regeneration while also improving RGC survival. However, whereas low concentrations of ZX1 are particularly effective in promoting regeneration (FIG. 7), all concentrations between 10 μM and 10 mM are roughly equally effective in promoting cell survival. Several other treatments similarly demonstrate a dissociation between RGC survival and axon regeneration (Chierzi et al. (1999) J Neurosci 19:8367-8376; Lorber et al., (2009) Nat Neurosci 12:1407-1414), and it seems likely that the effect of blocking extracellular ionic Zn²⁺ on RGC survival and axon regeneration involve different targets.

### Metal Chelator (ZX1) Promotes Retinal Ganglion Cells (RGC) Survival

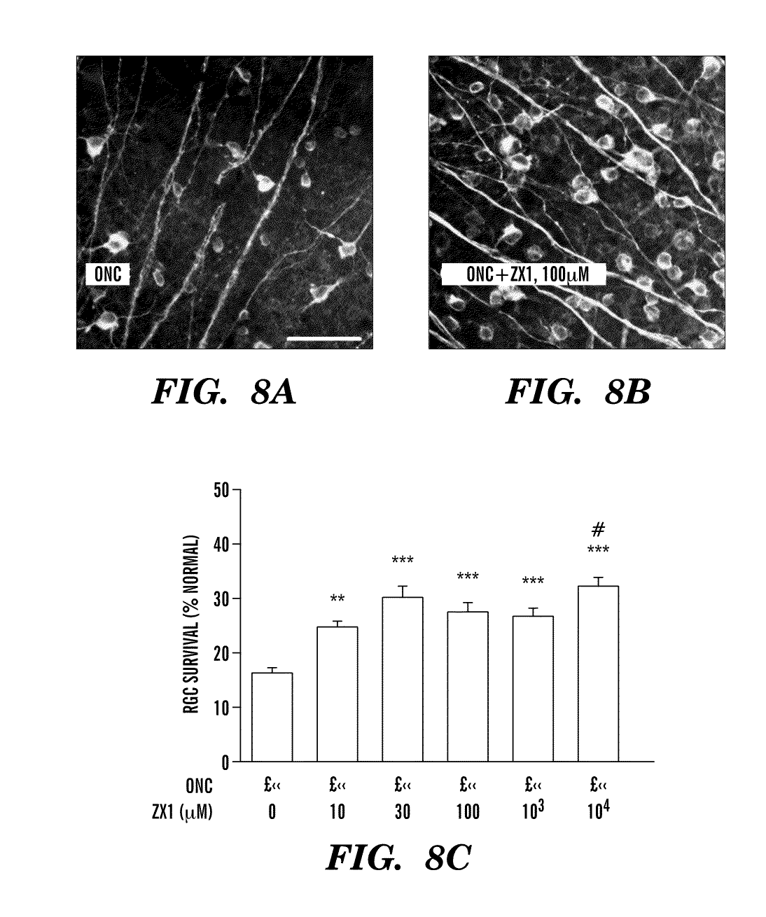

14 days after surgery, when compared to control group (only 16.35±0.89% RGC survived), a wide dose range of metal chelator (ZX1, 10 μM˜10 mM) had a significant effect on promoting retinal ganglion cells survival (24.65±1.21%˜32.17±1.73%). There was no significant dose response among groups except between 10 μM and 10 mM (p<0.05). (FIG. 8)

### Metal Chelator (ZX1) Enhances RGC Axon Regeneration

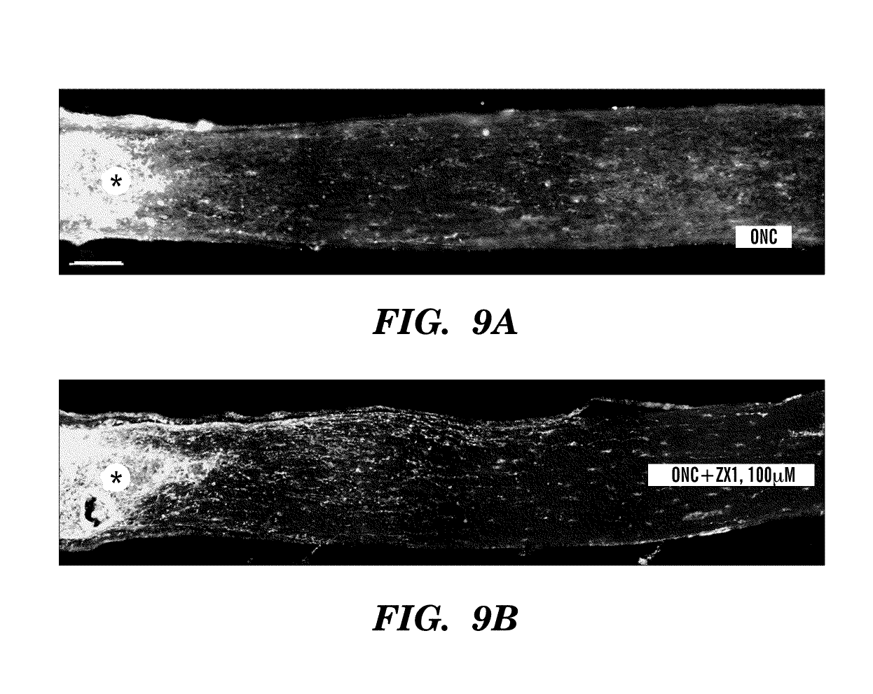

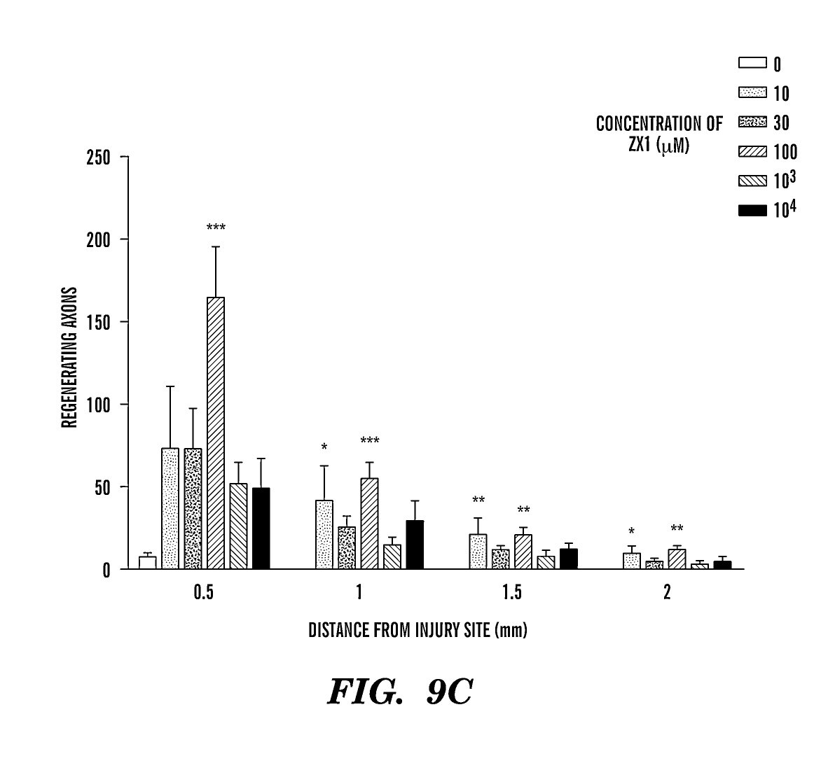

RGCs only express GAP-43 during axon outgrowth. Probes for this protein enable us to visualize regenerating axons. Animals with optic nerve crush alone averaged 7.05±2.49 axons extending 0.5 mm past the injury site, and none at longer distance. Axons in animals which received ZX1 (10 μM˜10 mM) injection showed strong outgrowth capabilities (up to 164) at 0.5 mm from crush site. ZX1 at 100 μM was the most effective concentration for intraocular injection based on our results. (FIG. 9)

### Materials and Methods

Animals.

Studies were performed at Children's Hospital Boston with the approval of the Institutional Animal Care and Use Committee. Experiments used male mice of the strain C57BL/6J (Jackson lab, Bar Harbor, Me.), at 8- to 10-week-old age.

Surgery.

Adult mice were anesthetized with a combination of ketamine and xylazine given intraperitoneally. A conjunctival incision was made over the dorsal aspect of one eye, which is then gently rotated downward in the orbit. The orbital muscles were slightly separated to expose the optic nerve at its exit from the globe, which were then crushed for 5 seconds with jewelers' forceps (Dumont number 5) near the back of the eye (within 0.5 mm). 3 μL ZX1 (10 μM˜10 mM in PBS,) was injected into the vitreous right after optic nerve crush and 4 days after injury, with care taken to avoid injuring the lens. Same volume PBS was injected intravitreally in control group.

Evaluating Optic Nerve Regeneration and Retina Ganglion Cells (RGC) Survival.

Mice were sacrificed with an overdose of anesthesia 14 d after optic nerve injury and were perfused with saline and 4% paraformaldehyde (PFA). These mice were 8 weeks old when sacrificed. Optic nerves and eyes were dissected and postfixed in PFA. Nerves were impregnated with 30% sucrose, embedded in OCT Tissue Tek Medium (Sakura Finetek), frozen, cut in the longitudinal plane at 14 μm, and mounted on coated slides. Regenerating axons were visualized by staining with a sheep antibody to GAP-43 followed by a fluorescently labeled secondary antibody.

Axon growth was quantified by counting the number of GAP-43-positive axons extending 0.5, 1, 1.5 and 2 mm from the end of the crush site in at least eight sections per case. The cross-sectional width of the nerve was measured at the point at which the counts were taken and was used to calculate the number of axons per millimeter of nerve width. The number of axons per millimeter was then averaged over the number of sections. Σa_{d}, the total number of axons extending distance d in a nerve having a radius of r, was estimated by summing over all sections having a thickness t (14 μm):

Σa_{d}=πr²×[average axons/mm]/t

RGC survival was evaluated in flat-mounted retinas immunostained with a rabbit antibody to βIII-tubulin (1:500; Abcam), followed by a secondary antibody made in goat and conjugated to Alexa Fluor 594, taking advantage of the selective expression of βIII-tubulin in RGCs. Images of eight preselected areas, 2 mm from the optic disc were captured under fluorescent illumination (400×; E800; Nikon). βIII-Tubulin-positive cells were counted using NIH ImageJ software (Wayne Rasband, National Institutes of Health, Bethesda, Md.). Cell densities were averaged across all eight areas.

Statistics.

Data are represented as means±SEM. Data analysis was performed using GraphPad Prism 5 software (GraphPad Software Inc., San Diego, Calif.). Statistical significance was determined using unpaired Student's t tests and one-way analysis of variance with ANOVA test.

### Example 2

### Zinc Chelators

The following shows the structure of various zinc chelators appropriate for use in the methods described herein.

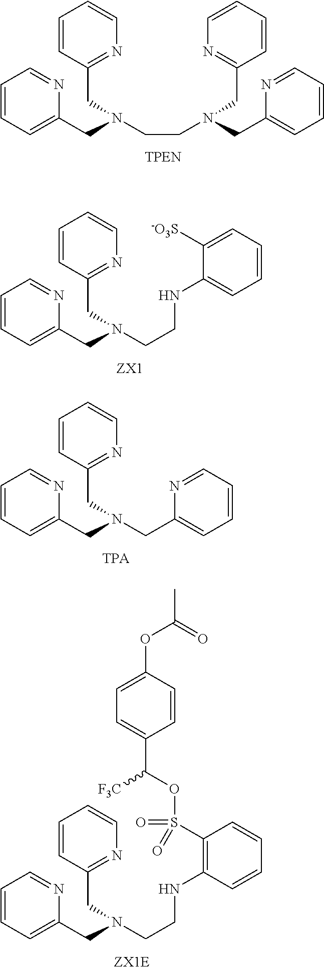

TPEN and TPA are commercially available. The synthesis of ZX1 (2-((Bis(pyridin-2-ylmethyl)amino)methylamino)benzenesulfonic acid) is provided in Pan, et al., Neuron 2011, 71, 1116-1126.

### Synthesis of ZX1E—a Trappable Zinc Chelator

Zinc-selective chelators are largely categorized into two classes: membrane permeable (e.g., TPEN, TPA) and impermeable (e.g., CaEDTA, tricine). Although impermeable chelators are uniquely suited for sustaining low concentrations of extracellular zinc (Pan, et al., Neuron 2011, 71, 1116-1126) permeable chelators readily diffuse out of the cell. A chelator that can be trapped inside cells would offer many advantages. Trappabililty may be achieved by capping the negative charges of acids of an impermeable chelator by an ester to render the molecule membrane-permeable (McQuade, et al., Inorg. Chem. 2010, 49, 9535-9545). The resultant molecule, once inside the cells, may be hydrolyzed to the corresponding acid and thereby become trapped inside the cells. An ideal trappable chelator should be stable in the media outside the cells, and once inside the cells become efficiently converted to the acid form by endogenous esterases. The acid form should also be soluble enough to achieve sufficiently high concentration inside the cells to make it useful. Based on these thoughts, we sought a trappable chelator by esterficiation of the sulfonic acid group of ZX1, an extracellular zinc-selective chelator recently developed. Unlike the carboxylic ester counterparts, however, many sulfonate esters are labile enough to become hydrolyzed in aqueous media. Recently trifluoromethylbenzyl (TFMB) group has been reported for protection of sulfonic acid (Rusha, L., Miller, S. C. Chem. Commun. 2011, 47, 2038-2040). The corresponding sulfonates is stable toward nucleophilic attack under neutral conditions due to the electronic effect of the β-fluoride moiety. In the presence of an esterase, cleavage of the acetate in the AcOTFMB moiety results in loss of 4-(2,2,2-trifluoro-1-hydroxyethyl)phenol to generate the corresponding sulfonic acid. The AcOTFMB group has been applied to cap sulfonic acid groups on fluorophores to afford the trappable versions (Rusha, L., Miller, S. C. Chem. Commun. 2011, 47, 2038-2040).

The synthesis of ZX1E, a trappable zinc chelator, 3, and preliminary characterizations of its trappability is described below.

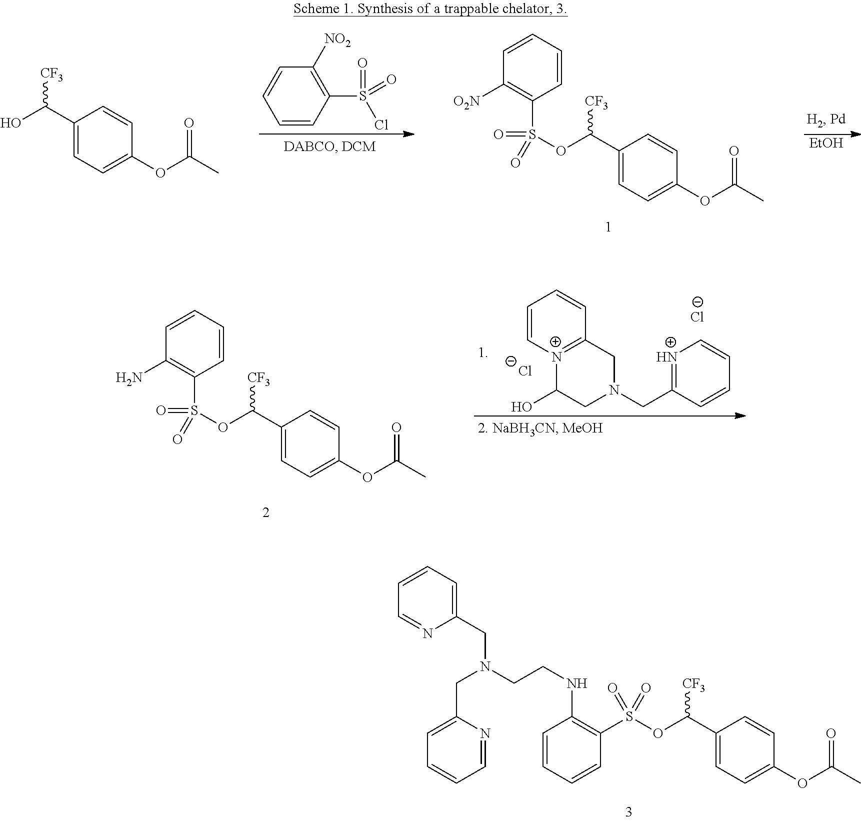

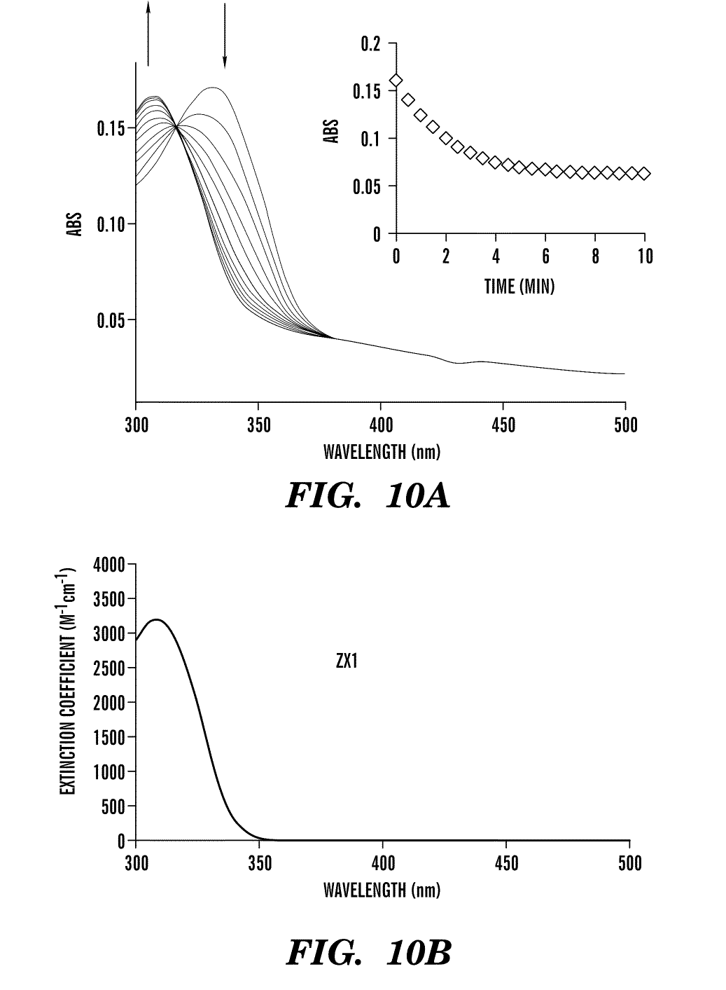

The AcOTFMB sulfonate ester, 1, was synthesized with 88% yield by esterifying 2-nitrobenzenesulfonyl chloride with the corresponding alcohol in the presence of DABCO as the base. Reduction of the nitro functionality with hydrogen afforded the aniline, 2, with 99% yield. The dipicolylamine (DPA) zinc-binding unit can be installed to the aniline via reduction amination to afford the target molecule, 3, in 10% yield (Scheme 1). 3 is stable at pH 7 for several days without being hydrolyzed, but is susceptible to enzymatic cleavage to afford ZX1. In 25% MeOH/PBS at 37° C., upon introduction of 0.2 unit/ml porcine liver esterase (PLE), The UV/vis spectrum gradually changes with an isosbestic point of 317 nm, to produce a peak at 307 nm (FIG. 10).

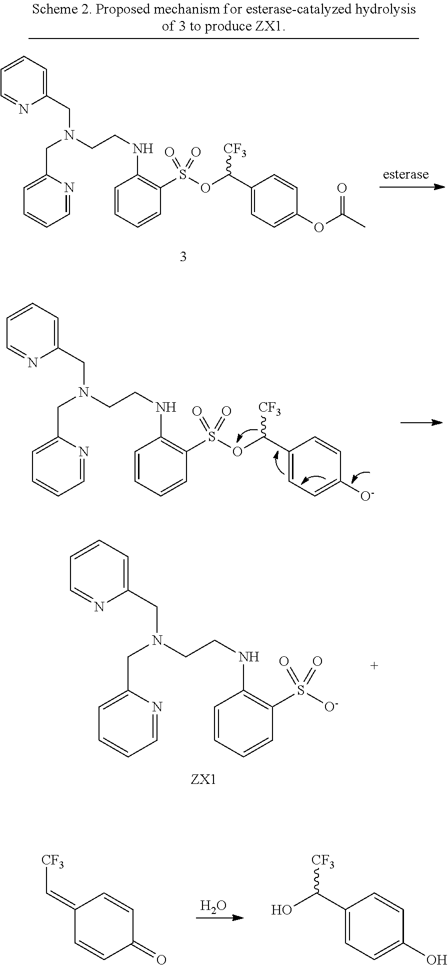 [SMILES: CC(=O)Oc1ccc(C(OS(=O)(=O)c2ccccc2NCCN(Cc2ccccn2)Cc2ccccn2)C(F)(F)F)cc1 ; O=S(=O)(OC(c1ccc([O-])cc1)C(F)(F)F)c1ccccc1NCCN(Cc1ccccn1)Cc1ccccn1 ; O=S(=O)([O-])c1ccccc1NCCN(Cc1ccccn1)Cc1ccccn1 ; O=C1C=CC(=CC(F)(F)F)C=C1 ; O ; Oc1ccc(C(O)C(F)(F)F)cc1]

### Materials and Methods

4-(2,2,2-trifluoro-1-hydroxyethyl)phenyl acetate and 4-hydroxy-2-(pyridin-1-ium-2-ylmethyl)-1,2,3,4-tetrahydropyrido[1,2-a]pyrazin-5-ium dichloride were prepared according to published procedures (25,28). Phosphate buffered saline (PBS) was purchased from Mediatech. All other materials were purchased from commercial suppliers and used as received.

Silica gel (SiliaFlash F60, Silicycle, 230-400 mesh) was used for column chromatography. Analytical thin layer chromatography (TLC) sheets were purchased from Mallinkrodt Baker, Inc. Reverse phase C18 preparative TLC plates were purchased from AnalTech Inc.

Deuterated NMR solvents were purchased from Cambridge Isotope Labs and used as received ¹H, ¹³C{¹H} NMR and ¹⁹F spectra were acquired on a Varian 300 or 500 MHz spectrometers at ambient temperature (283 K). Chemical shifts are reported in parts per million (δ) and are referenced to residual protic solvent resonances (for ¹H and ¹³C{¹H}) or external standard (CFCl₃ for ¹⁹F). The following abbreviations are used in describing NMR couplings: (s) singlet, (d) doublet, (dd) doublet of doublets, (t) triplet, (q) quartet, (m) multiplet, (br. s.) broad singlet. Low-resolution mass spectra were acquired on an Agilent 1100 Series LC/MSD Trap spectrometer.

### Synthetic Procedures

### 4-(2,2,2-trifluoro-1-(((2-nitrophenyl)sulfonyl)oxy)ethyl)phenyl acetate (shown above in scheme 1 as “1”)

4-(2,2,2-trifluoro-1-hydroxyethyl)phenyl acetate (1.20 g, 5.1 mmol) and 1,4-diazabicyclo[2.2.2]octane (DABCO, 0.57 g, 5.1 mmol) were combined in 5 mL of anhydrous dichloromethane under nitrogen. The colorless solution was cooled to 0° C., and 2-nitrobenzenesulfonyl chloride (2.42 g, 7.7 mmol) was added in small portions. The mixture was stirred at 0° C. and was allowed to warm up to room temperature. After 16 h, the white solid was removed by filtration, and 40 mL of dichloromethane was added to the filtrate. The solution was washed with 2×40 mL of water, dried with MgSO4, and the solvent was evaporated to give a yellow solid. Recrystallization with 600 mL of 1:2 diethyl ether/pentane afforded colorless needle crystals (1.72 g, 88% yield). 1H NMR (300 MHz, CDCl₃) δ 2.28 (s, 3H), 5.89 (q, J=6.1 Hz, 1H), 7.03 (d, J=8.8 Hz, 2H), 7.45 (d, J=8.4 Hz, 2H), 7.61 (m, 1H), 7.74 (d, J=3.3 Hz, 2H), 7.89 (d, J=7.7 Hz, 1H). ¹³C{¹H} NMR (126 MHz, CDCl₃) δ 21.2, 79.3 (q, J=35 Hz), 122.1 (q, J=281 Hz), 122.2, 125.0, 126.4, 129.6, 129.8, 131.3, 132.6, 135.5, 152.5, 169.0. ¹⁹F NMR (282 MHz, CDCl₃) δ −76.11 (d, J=6.1 Hz). MS (ESI): calcd [M+H]⁺, 420.0. found, 441.9.

### 4-(1-(((2-aminophenyl)sulfonyl)oxy)-2,2,2-trifluoroethyl)phenyl acetate (shown above in scheme 1 as “2”)

4-(2,2,2-trifluoro-1-(((2-nitrophenyl)sulfonyl)oxy)ethyl)phenyl acetate (1; 1.60 g, 3.82 mmol) and 10% palladium on carbon (61 mg) were combined in 110 mL of ethanol and purged with nitrogen for 15 min. A hydrogen atmosphere was applied, and the mixture was stirred at room temperature for 17 h. The system was purged with nitrogen and filtered, and solvent was removed under vacuum. The resultant yellow solid was re-dissolved in 300 mL of 1:2 diethyl ether/pentane and kept overnight at −20° C. The mixture was filtered, and the filtrate was concentrated under vacuum to give a light grey solid (1.47 g, 99% yield). ¹H NMR (500 MHz, CDCl₃) δ 2.28 (s, 3H), 4.82 (br. s., 2H), 5.55 (q, J=6.1 Hz, 1H), 6.55 (d, J=8.2 Hz, 1H), 6.61 (t, J=7.6 Hz, 1H), 6.99 (d, J=8.5 Hz, 2H), 7.22 (t, J=7.8 Hz, 1H), 7.31 (d, J=8.5 Hz, 2H), 7.50 (d, J=8.2 Hz, 1H). ¹³C{¹H} NMR (126 MHz, CDCl₃) δ 21.2, 77.5 (q, J=34.4 Hz), 116.2, 117.2, 117.6, 121.9, 122.2 (q, J=281 Hz), 126.9, 129.4, 130.0, 136.0, 146.4, 152.1, 169.0. 19 F NMR (282 MHz, CDCl₃) δ −76.33 (d, J=6.1 Hz). MS (ESI): calcd [M+Na]+, 412.0. found, 412.0.

### 4-(1-(((2-((2-(bis(pyridin-2-ylmethyl)amino)ethyl)amino)phenyl)sulfonyl)oxy)-2,2,2-trifluoroethyl)phenyl acetate (shown above in scheme 1 and 2 as “3”)

4-(1-(((2-aminophenyl)sulfonyl)oxy)-2,2,2-trifluoroethyl)phenyl acetate (2; 137 mg, 0.35 mmol), 4-hydroxy-2-(pyridin-1-ium-2-ylmethyl)-1,2,3,4-tetrahydropyrido[1,2-a]pyrazin-5-ium dichloride (122 mg, 0.39 mmol), and Na₂SO₄ (2.5 g, 18 mmol) were combined in 6 mL of anhydrous methanol under nitrogen, and were stirred at room temperature for 19 h. NaBH₃CN (89 mg, 1.4 mmol) were added, and the mixture was stirred for an additional 3 h. The solid were filtered off, and after addition of 20 mL of water, the solution was extracted with 2×20 mL of dichloromethane. The organic phase was dried with MgSO4, and concentrated under vacuum to a yellow oil. Purification of a fraction of the oil with reverse phase preparative TLC with methanol afforded the product as light yellow solid (5 mg, ca. 10% yield). ¹H NMR (300 MHz, CDCl₃) δ 2.26 (s, 3H), 2.87 (m, 3H), 3.11 (m, 1H), 3.78 (m, 2H), 3.94 (m, 2H), 5.25 (q, J=6.4 Hz, 1H), 6.20 (br. s., 1H), 6.28 (d, J=8.5 Hz, 1H), 6.55 (t, J=7.4 Hz, 1H), 6.80 (q, J=8.8 Hz, 4H), 7.20 (m, 3H), 7.60 (dd, J=8.1, 1.5 Hz, 1H), 7.77 (m, 4H), 8.53 (d, J=4.6 Hz, 2H). ¹⁹F NMR (282 MHz, CDCl₃) δ −76.22 (d, J=6.1 Hz). MS (ESI): calcd [M+H]⁺, 615.2. found, 615.4.

### Example 3

We have discovered a previously unknown, strong connection between zinc and axon regeneration. Our preliminary data show that levels of ionic Zn²⁺ increase dramatically in the dendritic field of retinal ganglion cells (RGCs) shortly after injury to the optic nerve, and that chelating Zn²⁺ promotes axon regeneration. The inability of neurons to regenerate axons after CNS injury, coupled with the low potential of undamaged neurons to form compensatory connections, results in life-long disabilities in victims of spinal cord injury, stroke, traumatic brain injury, optic nerve damage, and certain neurodegenerative disorders. Together, these conditions affect millions of people worldwide, and treatments to promote regeneration could therefore improve quality of life and reduce the economic burden for numerous patients, families, and society. Research over the past 20 years has shown that counteracting cell-extrinsic inhibitors of growth, activating neurons' intrinsic growth state, elevating cAMP and/or trophic factors, physiological activity, and bridges of biopolymers, stem cells and/or glia promote regenerative growth in animal models (1-9). However, the recovery achieved in these studies has so far been limited and no treatments are available yet to improve outcome clinically. The studies discussed herein begin to fill a major gap in our understanding of regenerative failure and identify Zn²⁺ dyshomeostasis as an early event.

The optic nerve has been widely used as a model to study CNS regeneration due to its accessibility, well-defined anatomy, and functional importance. RGCs, the projection neurons of the eye, cannot regrow injured axons and begin to die a few days after optic nerve damage, precluding visual recovery (10, 11). RGC death involves changes in dual leucine kinase and its downstream effectors (12, 13), ER stress and the unfolded pro-tein response (14), oxidative damage (15), diminished intracellular cAMP (16, 17), increases in NO (18), caspase activation (19, 20), and changes in the expression of anti- and pro-apoptotic Bcl-like proteins (21). Most attempts to block these changes delay, but do not prevent, RGC loss, and the initial event that triggers death is unknown. Furthermore, although enhancing RGC survival is clearly a precondition for regeneration, additional factors are required to promote axon growth per se (22, 23). One such factor is Oncomodulin (Ocm), a potent growth factor for RGCs produced by cells of the innate immune system (11, 24-28). Regeneration can also be induced by deleting the pten gene to de-repress signaling through the PI3 kinase pathway (29). Com-bining pten deletion with Ocm from inflammatory cells and a cAMP analog has a strong synergistic effect (11), enabling some RGCs to regenerate axons from the eye into central target areas, where they form synapses and restore some visual responses (30). Yet while these studies show the feasibility of full-length regeneration, ⅔ of RGCs still die after nerve damage, and <10% of the surviving RGCs go on to regenerate their axons (30). Thus, we need to identify other major regulators of cell survival and axon regeneration. Chelating free Zn²⁺ promotes both processes through mechanisms likely to be distinct from one another. These studies are the first to explore the role of Zn²⁺ as a regulator of axon regeneration. The role of Zn²⁺ in regulating axon growth has not been recognized before, nor has the role of synaptic changes. Understanding how Zn²⁺ regulates regeneration and cell survival will allow us to improve outcome after nerve damage.

Zn²⁺ is tightly bound to many proteins in the CNS, but also may exist in ionic form within synaptic vesicles and other intracellular organelles. Ionic Zn²⁺ can be liberated from metallothionein and intracellular organelles, or can enter cells through voltage-gated Ca²⁺ channels, Ca²⁺-permeable glutamate receptors, and/or specific Zn²⁺ transporters. Besides its normal functions, Zn²⁺ is a major factor in ischemic and traumatic brain damage, and very likely in other CNS disorders as well (31-34). Our preliminary data show a dramatic rise in ionic Zn²⁺ in synaptic layers of the retina that are rich in the Zn²⁺ transporter ZnT3, followed by a delayed accumulation in RGCs.

### Characterize the Timing, Localization, and Mechanism of Zn²⁺ Accumulation Following ONC

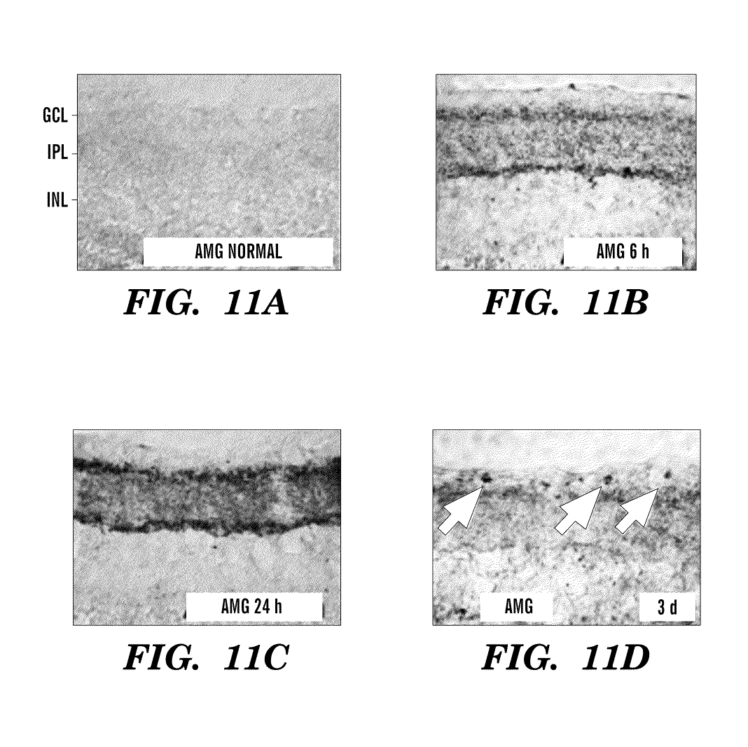

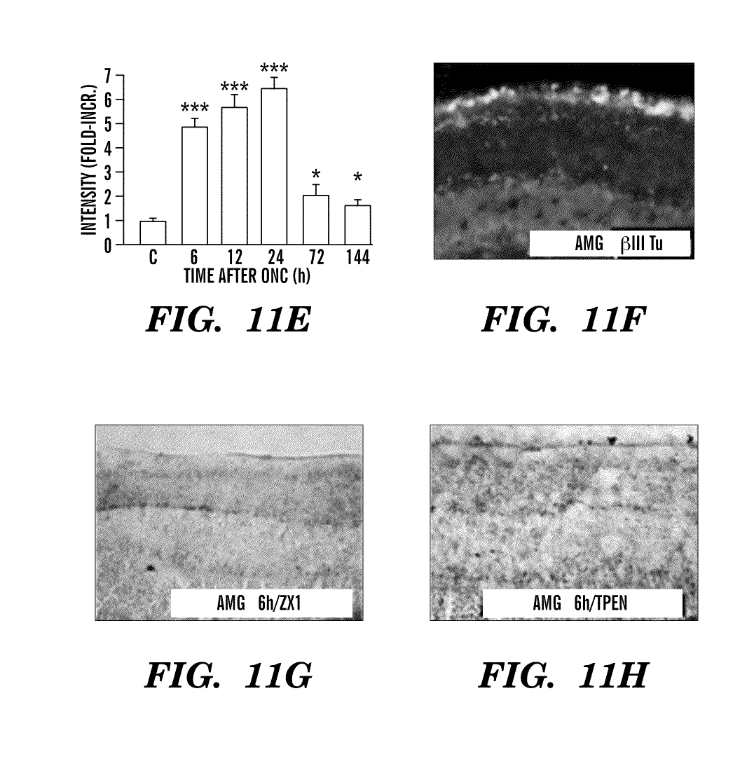

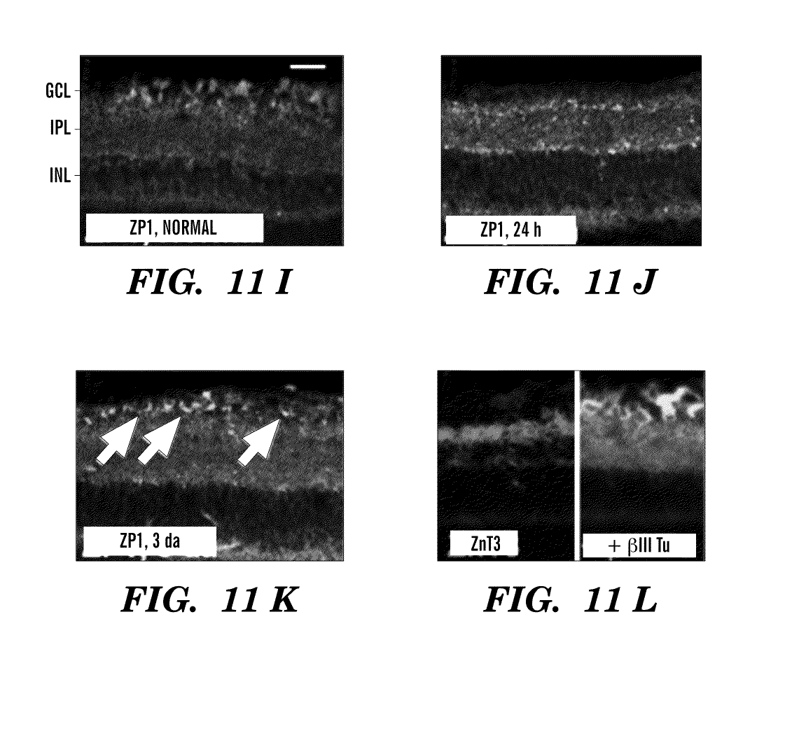

To investigate the possible relationship between ionic Zn²⁺ and nerve regeneration, we used autometallography (AMG) to examine changes in the retina, injecting Na2SeO3 i.p. at various times after ONC and euthanizing mice 6 hr later. We detected a dramatic increase in labeling in the IPL, the layer of the retina that contains synaptic inputs from retinal interneurons onto RGC dendrites, especially in 2 sublayers (FIG. 11B). IPL labeling increased up to 24 hr but declined by 3 days, at which time labeling appeared in RGCs themselves. ZX1, a highly specific, membrane-impermeable Zn²⁺ chelator (56), or TPEN (N,N,N′,N′-tetrakis(2-pyridyl methyl)ethylenediamine), a membrane-permeable chelator (40), blocked injury-induced labeling in the IPL (FIG. 11G, H), suggesting that the AMG signal represents Zn²⁺ per se. This interpretation is supported by studies using a membrane-permeable fluorescent Zn²⁺ sensor, Zinpyr1, which has a strong preference for Zn²⁺ over Ca²⁺ (41). Zinpyr1 revealed labeling in the IPL 6 hr after nerve damage and this remained strong at 24 hr (FIG. 11J). As with AMG, Zinpyr1 labeling was elevated in RGCs but not IPL at 3 days (FIG. 11K). The Zn²⁺ transporter ZnT3 is also localized in the IPL (FIG. 11L).

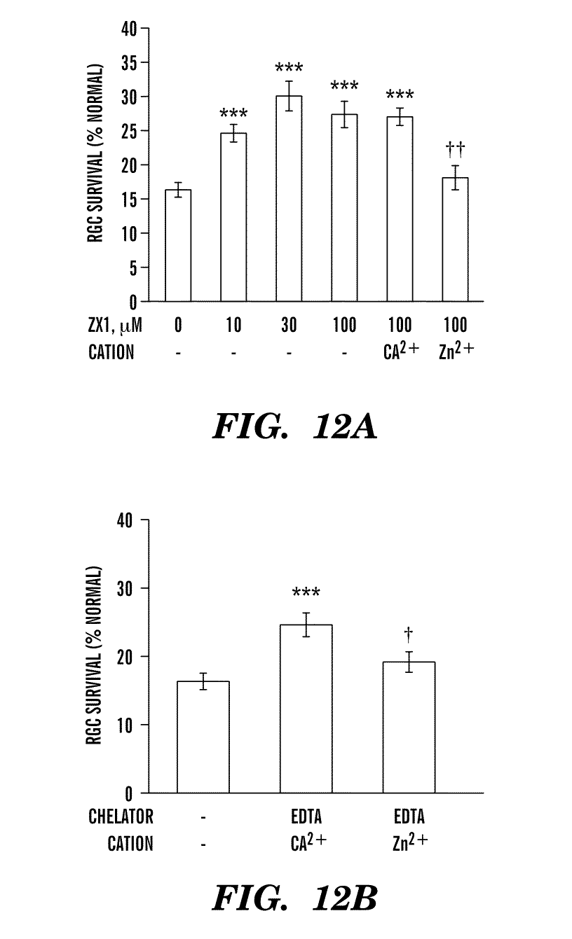

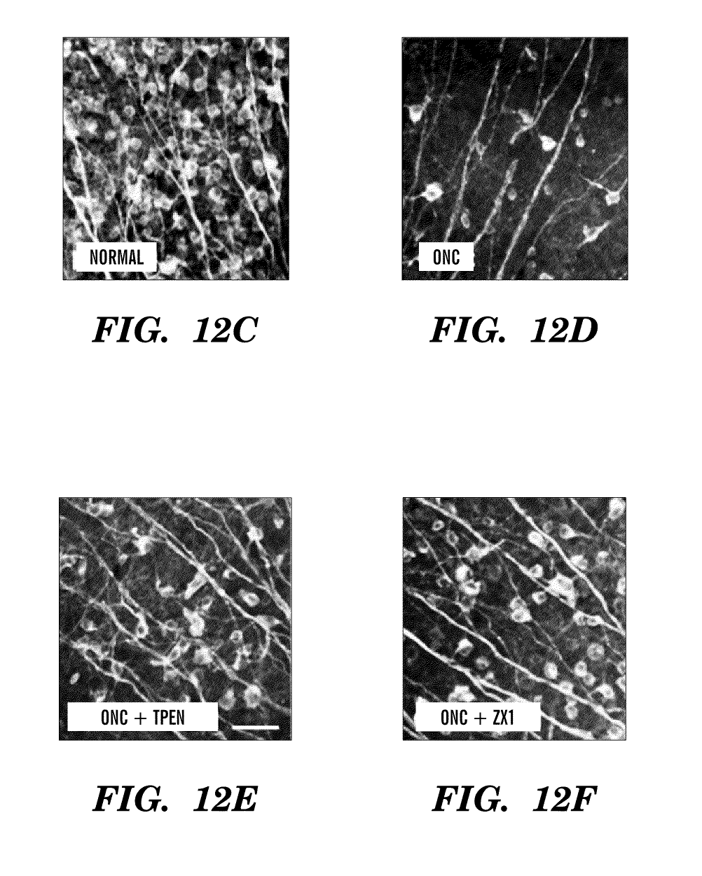

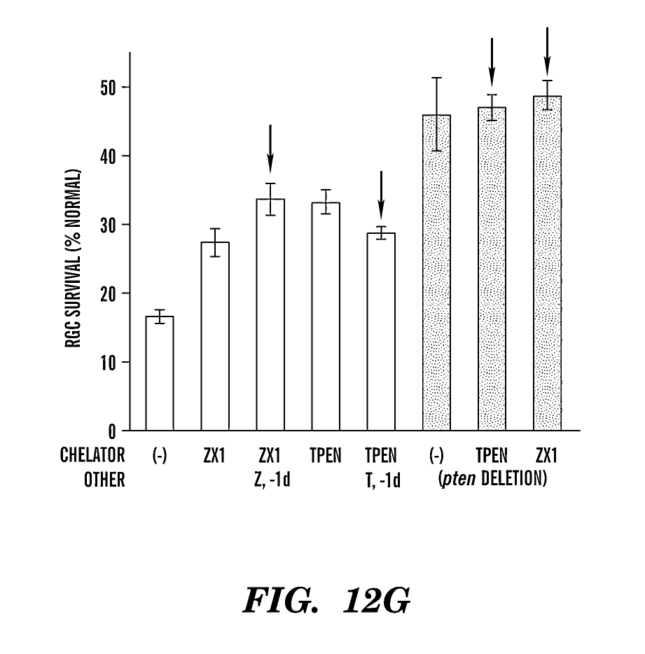

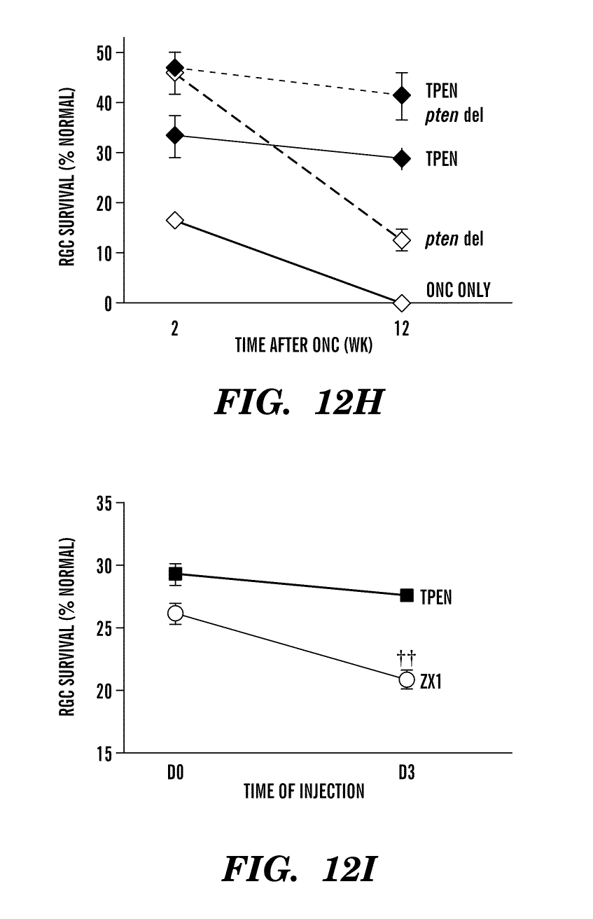

We next investigated the functional significance of Zn²⁺ accumulation. Injecting ZX1 or CaEDTA into the eye just after ONC and again after 4 days doubled the number of RGCs that survived 2 weeks after ONC (FIG. 12). The specificity of this effect was demonstrated by the finding that saturating either chelator with equimolar Zn²⁺, (but not Ca²⁺), abrogated their effects (FIG. 12A&B). TPEN (100 μM) also protected RGCs, and this effect was likewise blocked by pre-saturating with Zn²⁺ (not shown; P<0.01). The effect of Zn²⁺ chelation on RGC survival, though partial, endured for months (FIG. 12H). In contrast, caspase inhibition (42, 43), trophic factors (44, 45), blocking the unfolded protein response (14), or deleting the pten gene (FIG. 12H) (29) confer only transient protection. This implies that Zn²⁺ may be a “master regulator” of RGC death that, once blocked, prevents activation of other death pathways. Because Zn²⁺ does not accumulate in RGCs until 2-3 da, we tested whether a chelator given only on Day 3 would be protective. The membrane-permeable chelator TPEN was fully protective if given on Day 3 (FIG. 12I), though the membrane-impermeable chelator ZX1 was less effective (FIG. 12I). Thus, late-stage protection may require access to intracellular Zn²⁺.

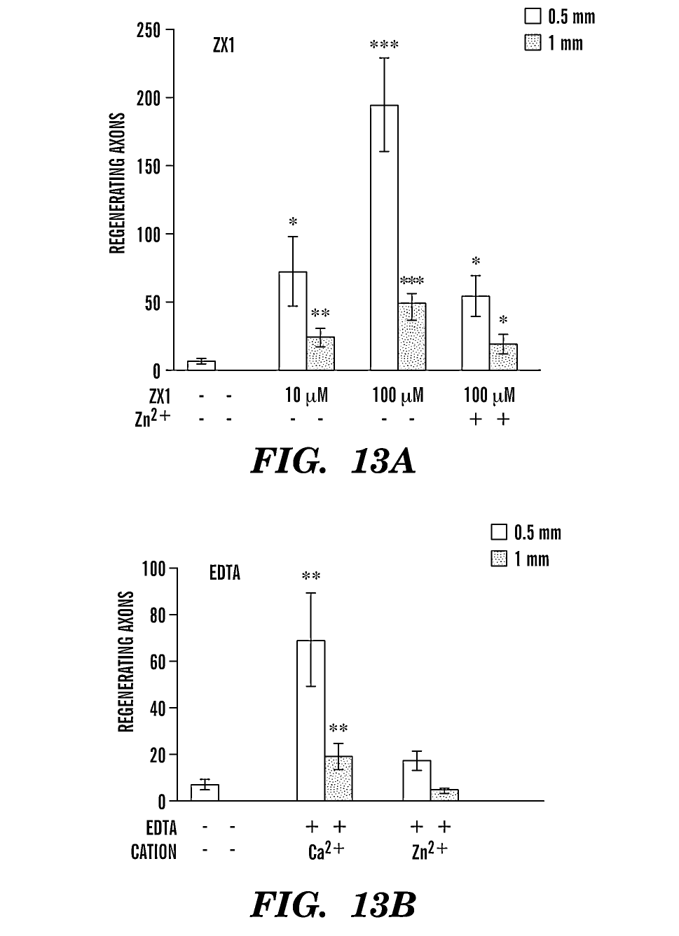

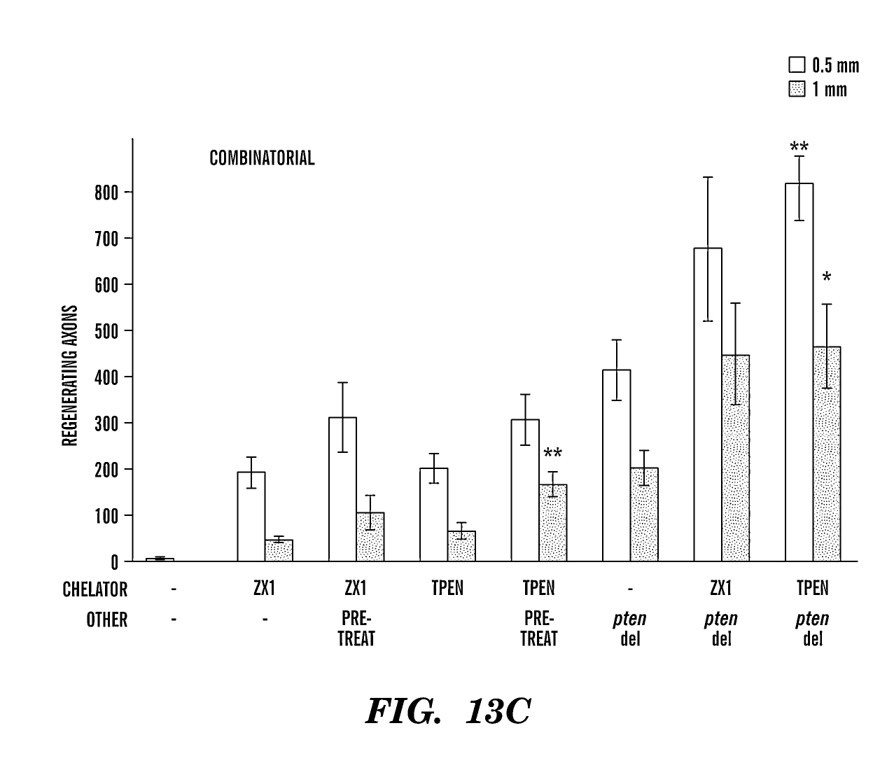

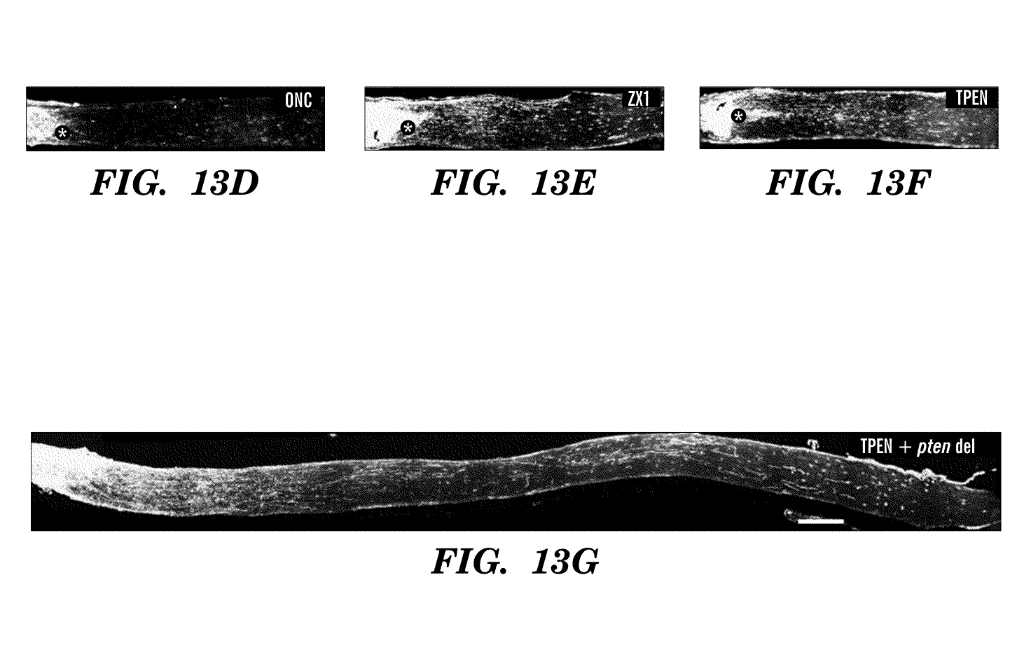

Unexpectedly, Zn²⁺ chelation stimulated axon regeneration. ZX1 achieved a maximal effect at 100 μM (FIG. 13). As with survival, this effect appears to be related to Zn²⁺ per se, and was eliminated by saturating ZX1 with Zn²⁺ (FIG. 13A). Intraocular injection of CaEDTA, but not ZnEDTA, also led to axon outgrowth (FIG. 13B), as did TPEN (FIG. 13C, F). We next examined how the timing of chelation affected regeneration. ZX1 or TPEN injected 1 day before ONC augmented the amount of regeneration achieved by 2 post-injury injections (FIG. 13C) but did not enhance RGC survival (FIG. 12G). On the other hand, TPEN injected on D3 had the same effect on survival as administering it on days 0 and 4 (see FIGS. 12G and 12I), but did not promote regeneration. Thus, the effects of Zn²⁺ chelation on RGC survival and regeneration may involve distinct mechanisms. Finally, combining Zn²⁺ chelators with pten gene deletion enabled some axons to regenerate all the way to the optic chasm in 2 weeks (FIG. 13C,D-G). However, this combination did not augment survival (FIG. 12G).

### Zn²⁺ Accumulation in the IPL is Mediated by ZnT3 in Terminals of a Particular Type of Interneuron

An earlier study using AMG and electron microscopy (EM) reported that Zn²⁺ is present in a subset of retinal interneurons and their nerve terminals in the IPL that were thought to be dopaminergic amacrine cells due to the presence of dense-core vesicles (46). ZnT3 is a Zn²⁺ transporter found in glutamatergic (47) and aminergic (48) vesicles, and was reported to be present in the GCL and weakly in the IPL (49). Using a short post-fixation time, we observed high ZnT3 levels in terminals in the IPL having a similar distribution as the free Zn²⁺ seen after ONC (FIG. 11L). However, the ZnT3-positive structures in the normal IPL only become laden with free Zn²⁺ (FIGS. 11A,I) after nerve injury. We hypothesize that nerve injury causes Zn²⁺ to be released from a storage site such as metallothionein and to accumulate in and perhaps around ZnT3+ synaptic vesicles.

### Zn²⁺ Accumulates in Injured RGCs as a Consequence of Nitric Oxide (NO) Production

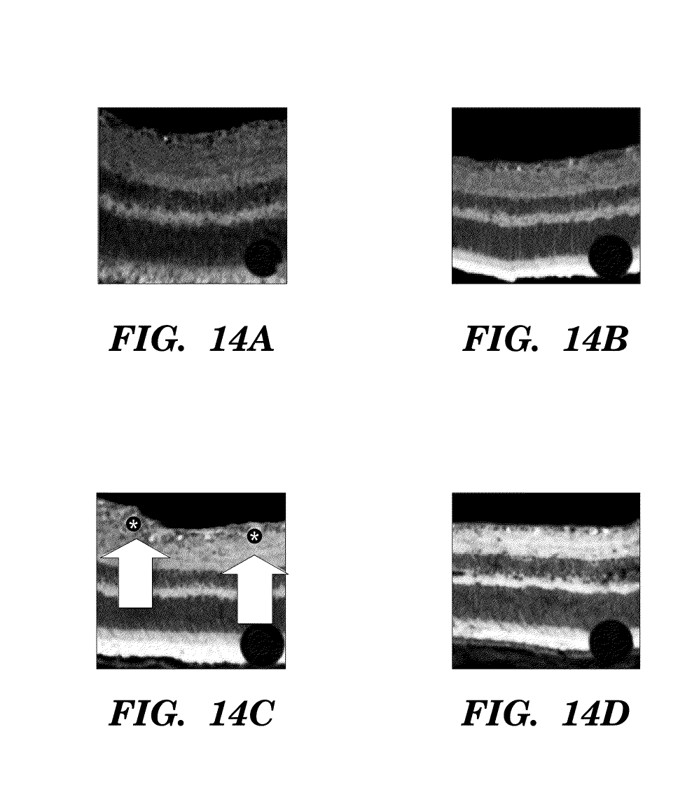

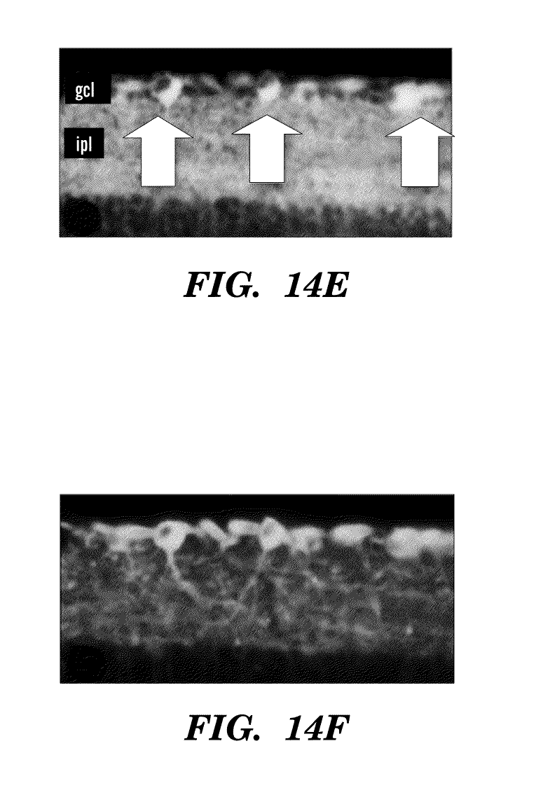

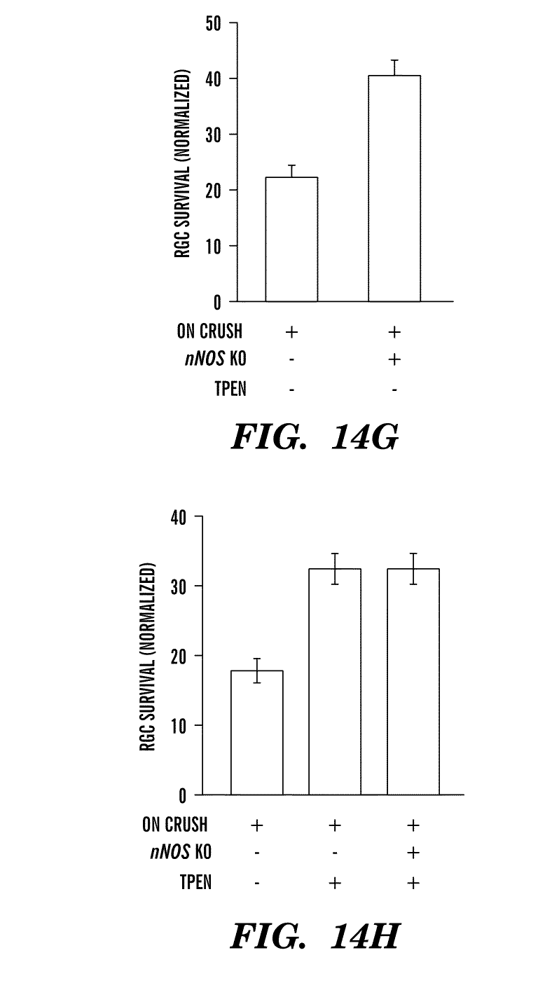

Zn²⁺ can be released from intracellular stores such as metallothioneins by oxidation of sulfhydryl groups on these proteins (62, 63). Previous studies have shown that the expression of neuronal NO synthase (nNOS) increases 3 days after ONC in the rat, peaks by 5 da, and returns to baseline by P28 (64). Using the NO sensor DAF-2, we first detect NO in mouse RGCs 3 days after ONC (FIG. 14C). Genetic knockout of nNOS is protective to RGCs following ONC (FIG. 14G). If the accumulation of Zn²⁺ in RGCs after ONC is due to NO production, then genetic knockout of nNOS should occlude the protective effect of TPEN. We have found this to be the case (FIG. 14H). To further test the idea that nNOS activation and NO are responsible for Zn²⁺ accumu-lation, we will detect mobile Zn²⁺ under conditions in which NO production is limited by nNOS knockout or pharmacological blockade of nNOS or by a scavenger that eliminates NO once it has been produced.

### Zn²⁺ Chelation Promotes Axon Regeneration and Cell Survival Through Distinct Mechanisms

Although the decline in RGC viability that occurs after optic nerve injury clearly limits the extent of axon regeneration, several studies suggest that RGC survival and axon regeneration involve distinct mecha-nisms (83). For example, Bcl-2 or Bcl-xL overexpression suppresses RGC death without promoting regenera-tion (22, 83, 84), whereas the growth factor Ocm promotes regeneration without enhancing survival (11). Blocking DLK promotes RGC survival after optic nerve injury but prevents RGCs from regenerating axons (13). Our preliminary time-course studies suggest that the effects of Zn²⁺ chelation on regeneration and survival also may involve distinct mechanisms. As noted, initiating chelation 24 h prior to ONC enhances axon regeneration but not survival (cf. FIGS. 12G and 13C). This observation suggests that (a) when mobile or free Zn²⁺ accumulates in the synaptic field of RGCs, it suppresses regeneration, and (b) this effect is distinct from the effect of Zn²⁺ in suppressing RGC survival.

### Zn²⁺ Chelation Promotes Axon Regeneration by a Non-Cell Autonomous Mechanism

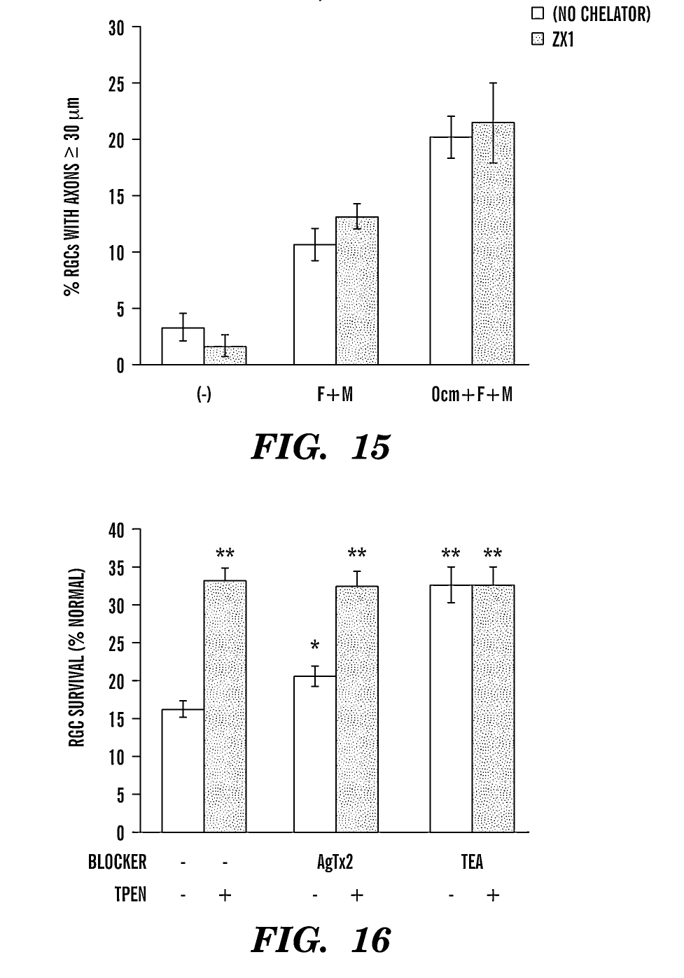

Our preliminary data suggest that the effect of Zn²⁺ in suppressing RGCs' ability to regenerate ax-ons may not be cell-autonomous. Examples of presumed cell-autonomous effects on RGCs include deleting the pten or klf4 gene in RGCs, or overexpressing a constitutively active form of the protein kinase Mst3b in these cells, all of which promote axon outgrowth (29, 85, 88). One important non-cell autonomous effect is the developmental decline in RGCs' capacity for robust axon growth due to interactions with amacrine cells (89). Although Zn²⁺ chelation strongly promotes axon regeneration in vivo, our preliminary data show that it does not cause adult RGCs to extend axons when they are dissociated in culture, nor does it enhance the effects of other growth-promoting signals (FIG. 15). This observation suggests that, in vivo, Zn²⁺ may suppress RGCs re-generative capacity indirectly by acting upon a cellular element that is absent in culture, for example synaptic contacts. This possibility is supported by the observation that chelating Zn²⁺ early on, when it is only present in the IPL, promotes regeneration, whereas chelation 3 days after ONC, when Zn²⁺ is localized in RGCs, has little effect on regeneration despite having a strong effect on cell survival (FIG. 12I). These data suggest that Zn²⁺ located in the IPL suppresses axon regeneration.

Chelating Zn²⁺ Augments the Effects of Other Pro-Regenerative Treatments and Improves Functional Outcome after Optic Nerve Damage

As noted above, a combination of methods that activate RGCs' intrinsic growth state enables some RGCs to regenerate axons to appropriate target sites and restore simple visual responses (30). However, most RGCs continue to die and only a small percentage of those that remain successfully regenerate their axons. We hypothesize that the inability of most RGCs to regenerate axons is due in part to elevation of Zn²⁺, and that chelating Zn²⁺ will augment recovery well beyond current levels. Our preliminary data indicate that Zn²⁺ chela-tion strongly enhances the effect of deleting pten (FIG. 13C,G) and of Zymosan combined with a cAMP analog. PTEN suppresses cell signaling through the PI3 kinase/Akt pathway; Zymosan induces infiltrative neutro-phils and macrophages to secrete Ocm, and cAMP enhances the binding of Ocm to its receptor (11, 26, 27).

Pathways by which Zn²⁺ Suppresses, and Chelation Enhanced, RGC Survival

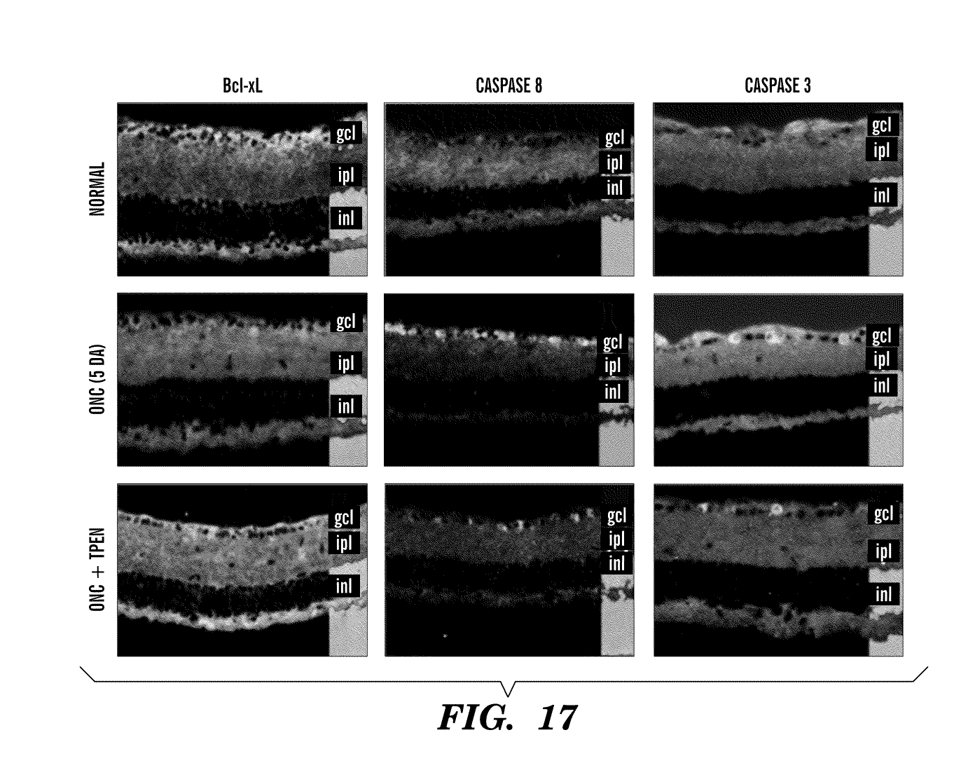

The death of RGCs after ONC is induced by a mechanism that involves nitric oxide (NO) production and Zn²⁺ accumulation, followed by K+ channel activation, caspase activation, and Bcl-xL degradation. Many possible roles for Zn²⁺ in cell death have been suggested (39), but one that has been particularly well characterized involves efflux of K+ and cell shrinkage (100) due to insertion of K+ channels into the cell membrane (101). Evidence for this and other pathways comes primarily from cell culture studies. The question of which pathways are actually involved in the death of RGCs following optic nerve injury in vivo will be addressed here (21). Our preliminary experiments (FIG. 16) suggest that 1) the K+ channel blocker tetraethylammonium (TEA) blocks RGC death after ONC to the same extent as TPEN; 2) TEA affords strong protection at ≦100 μM; 3) Agitoxin-2 (Ag-2), a blocker of Shaker family K+ channels Kv1.1 and 1.3, has only a small effect on RGC survival, as noted before (102); 4) TPEN occludes the effects of TEA and Ag-2.

We generally think of cell death as occurring by caspase-dependent apoptosis, caspase-independent programmed necrotic death, and necrosis (103). Bcl2 family members, including Bcl-xL, are important mediators and regulators of cell death (104). We observe that: 1) TPEN attenuates injury-induced activation of Caspases 8 and 3 in RGCs (FIG. 7); 2) a pan-caspase inhibitor and a caspase 8 inhibitor increase RGC survival (43, 105); 3) TPEN partially blocks Bcl-xL loss after ONC (FIG. 17). These results suggest that RGC death after ONC is associated with a rise in intracellular Zn²⁺ that increases the activity of one or more K+ channels with a high affinity for TEA; and that caspase activation and Bcl-xL degradation may lie downstream of Zn²⁺ elevation and K+ channel activation.

### Zinc Chelation Elevates Regeneration Above Previously Reported Methods

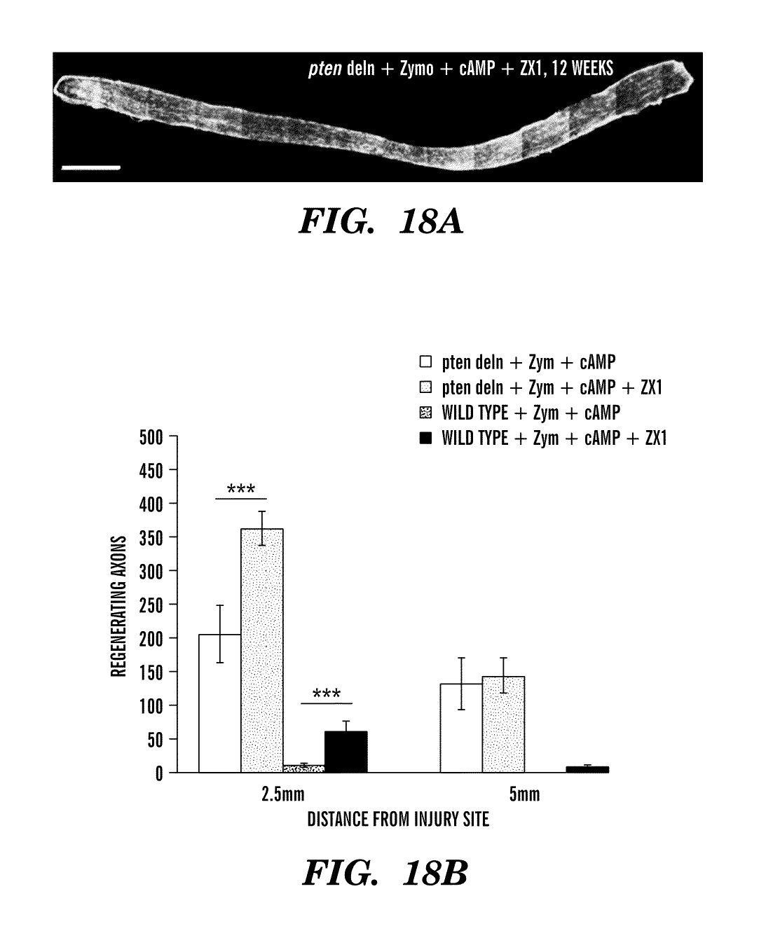

The long-term effect of combining the zinc chelator ZX1 with other activators of neuronal outgrowth, such as Zymosan, elevated cAMP and, pten gene deletion, are shown in FIG. 18. Studies from our group showed that a combination of Zymosan, elevation of cAMP, and deletion of the pten gene stimulates more extensive regeneration than ever reported before, and that this is accompanied by a partial return of function (30). Zymosan causes the entry of inflammatory cells into the eye that secrete the growth factor oncomodulin (Ocm). Elevation of cAMP increases the ability of Ocm to bind to its receptor. Deletion of the pten gene de-represses signaling through the PI3 kinase-Akt pathway. The addition of ZX1, a chelator of ionic zinc, nearly doubles the amount of regeneration seen at short distances from the injury site. ZX1 also increases the effect of Zymosan+cAMP alone. However, these latter effects are limited by the fact that the treatments are given only at the time of nerve injury and not continuously, whereas the effects of pten deletion endure. More extended administration is expected to promote regeneration to longer distances from the injury and/or administration site.

Even when examined 12 weeks after the chelator was administered, its effect is apparent for axons counted half-way down the nerve (end of the nerve is 5 mm). The results show that the combination of zinc chelation has a strong effect over and above the effects of the other agents in the absence of zinc chelation that were recently shown to provide unprecedented levels of regeneration.

### References Example 3

1. Schwab M E. Repairing the injured spinal cord. Science. 2002; 295:1029-31. PMID: 11834824
2. Carmeliet P, Tessier-Lavigne M. Common mechanisms of nerve and blood vessel wiring. Nature. 2005; 436:193-200. PMID: 16015319
3. Lee D H, Strittmatter S M, Sah D W. Targeting the Nogo receptor to treat central nervous system injuries. Nat Rev Drug Discov. 2003; 2:872-8. PMID: 14668808
4. Silver J, Miller J H. Regeneration beyond the glial scar. Nat Rev Neurosci. 2004; 5:146-56. PMID: 14735117
5. Hannila S S, Siddiq M M, Filbin M T. Therapeutic approaches to promoting axonal regeneration in the adult mammalian spinal cord. Int Rev Neurobiol. 2007; 77:57-105. PMID: 17178472
6. Sun F, He Z. Neuronal intrinsic barriers for axon regeneration in the adult CNS. Curr Opin Neurobiol. 2010; 20:510-8. PMID: 20418094
7. Kadoya K, Tsukada S, Lu P, Coppola G, Geschwind D, Filbin M T, et al. Combined intrinsic and extrinsic neuronal mechanisms facilitate bridging axonal regeneration one year after spinal cord injury. Neuron. 2009; 64:165-72. PMID: 19874785
8. Lu P, Wang Y, Graham L, McHale K, Gao M, Wu D, et al. Long-distance growth and connectivity of neural stem cells after severe spinal cord injury. Cell. 2012; 150:1264-73. PMID: 22980985
9. van den Brand R, Heutschi J, Barraud Q, DiGiovanna J, Bartholdi K, Huerlimann M, et al. Restoring voluntary control of locomotion after paralyzing spinal cord injury. Science. 2012; 336:1182-5. PMID: 22654062
10. Berkelaar M, Clarke D B, Wang Y C, Bray G M, Aguayo A J. Axotomy results in delayed death and apoptosis of retinal ganglion cells in adult rats. J Neurosci. 1994; 14:4368-74.
11. Kurimoto T, Yin Y, Omura K, Gilbert H Y, Kim D, Cen L P, et al. Long-distance axon regeneration in the mature optic nerve: contributions of oncomodulin, cAMP, and pten gene deletion. J Neurosci. 2010; 30:15654-63. PMID: 21084621
12. Welsbie D S, Yang Z, Ge Y, Mitchell K L, Zhou X, Martin S E, et al. Functional genomic screening identifies dual leucine zipper kinase as a key mediator of retinal ganglion cell death. Proc Natl Acad Sci USA. 2013; 110:4045-50. PMID: 23431148
13. Watkins T A, Wang B, Huntwork-Rodriguez S, Yang J, Jiang Z, Eastham-Anderson J, et al. DLK initiates a transcriptional program that couples apoptotic and regenerative responses to axonal injury. Proc Natl Acad Sci USA. 2013; 110:4039-44. PMID: 23431164
14. Hu Y, Park K K, Yang L, Wei X, Yang Q, Cho K S, et al. Differential effects of unfolded protein response pathways on axon injury-induced death of retinal ganglion cells. Neuron. 2012; 73:445-52. PMID: 22325198
15. Yuki K, Ozawa Y, Yoshida T, Kurihara T, Hirasawa M, Ozeki N, et al. Retinal ganglion cell loss in superoxide dismutase 1 deficiency. Invest Ophthalmol V is Sci. 2011; 52:4143-50. PMID: 21421868
16. Meyer-Franke A, Wilkinson G A, Kruttgen A, Hu M, Munro E, Hanson M G, Jr., et al. Depolarization and cAMP elevation rapidly recruit TrkB to the plasma membrane of CNS neurons. Neuron. 1998; 21:681-93.
17. Corredor R G, Trakhtenberg E F, Pita-Thomas W, Jin X, Hu Y, Goldberg J L. Soluble adenylyl cyclase activity is necessary for retinal ganglion cell survival and axon growth. J Neurosci. 2012; 32:7734-44. PMID: 22649251
18. Koeberle P D, Ball A K. Nitric oxide synthase inhibition delays axonal degeneration and promotes the survival of axotomized retinal ganglion cells. Exp Neurol. 1999; 158:366-81. PMID: 10415143
19. Kermer P, Ankerhold R, Klocker N, Krajewski S, Reed J C, Bahr M. Caspase-9: involvement in secondary death of axotomized rat retinal ganglion cells in vivo. Brain Res Mol Brain Res. 2000; 85:144-50. PMID: 11146116
20. Kermer P, Klocker N, Labes M, Thomsen S, Srinivasan A, Bahr M. Activation of caspase-3 in axotomized rat retinal ganglion cells in vivo. FEBS Lett. 1999; 453:361-4. PMID: 10405176
21. Isenmann S, Kretz A, Cellerino A. Molecular determinants of retinal ganglion cell development, survival, and regeneration. Prog Retin Eye Res. 2003; 22:483-543. PMID: 12742393
22. Chierzi S, Cenni M C, Maffei L, Pizzorusso T, Porciatti V, Ratto G M, et al. Protection of retinal ganglion cells and preservation of function after optic nerve lesion in bcl-2 transgenic mice. Vision Res. 1998a; 38:1537-43.
23. Goldberg J L, Espinosa J S, Xu Y, Davidson N, Kovacs G T, Barres B A. Retinal ganglion cells do not extend axons by default: promotion by neurotrophic signaling and electrical activity. Neuron. 2002; 33:689-702.
24. Kurimoto T, Yin Y, Habboub G, Gilbert H-Y, Li Y, Nakao S, et al. Neutrophils express oncomodulin and promote optic nerve regeneration. J Neurosci. 2013; (under revision).
25. Kurimoto T, Yin Y, Gilbert H, Habboub G, Li Y, Hafezi-Moghadam A, et al. Neutrophils express oncomodulin and promote optic nerve regeneration in mice. Abstracts, Society for Neuroscience Annual Meeting. 2012; In press.
26. Yin Y, Cui Q, Gilbert H Y, Yang Y, Yang Z, Berlinicke C, et al. Oncomodulin links inflammation to optic nerve regeneration. Proc Natl Acad Sci USA. 2009; 106:19587-92. PMID: 19875691
27. Yin Y, Henzl M T, Lorber B, Nakazawa T, Thomas T T, Jiang F, et al. Oncomodulin is a macrophage-derived signal for axon regeneration in retinal ganglion cells. Nat Neurosci. 2006; 9:843-52. PMID: 16699509
28. de Lima S, Habboub G, Benowitz L I. Combinatorial therapy stimulates long-distance regeneration, target reinnervation, and partial recovery of vision after optic nerve injury in mice. Int Rev Neurobiol. 2012; 106:153-72. PMID: 23211463
29. Park K K, Liu K, Hu Y, Smith P D, Wang C, Cai B, et al. Promoting axon regeneration in the adult CNS by modulation of the PTEN/mTOR pathway. Science. 2008; 322:963-6. PMID: 18988856
30. de Lima S, Koriyama Y, Kurimoto T, Oliveira J T, Yin Y, Li Y, et al. Full-length axon regeneration in the adult mouse optic nerve and partial recovery of simple visual behaviors. Proc Natl Acad Sci USA. 2012; 109:9149-54. PMID: 22615390
31. Koh J Y, Suh S W, Gwag B J, He Y Y, Hsu C Y, Choi D W. The role of zinc in selective neuronal death after transient global cerebral ischemia. Science. 1996; 272:1013-6. PMID: 8638123
32. Land P W, Aizenman E. Zinc accumulation after target loss: an early event in retrograde degeneration of thalamic neurons. Eur J Neurosci. 2005; 21:647-57. PMID: 15733083
33. Sensi S L, Paoletti P, Bush A I, Sekler I. Zinc in the physiology and pathology of the CNS. Nature reviews. 2009; 10:780-91. PMID: 19826435
34. Frederickson C J, Koh J Y, Bush A I. The neurobiology of zinc in health and disease. Nat Rev Neurosci. 2005; 6:449-62. PMID: 15891778
35. Zhang Y, Aizenman E, DeFranco D B, Rosenberg P A. Intracellular zinc release, 12-lipoxygenase activation and MAPK dependent neuronal and oligodendroglial death. Mol Med. 2007; 13:350-5. PMID: 17622306
36. Zhang Y, Wang H, Li J, Dong L, Xu P, Chen W, et al. Intracellular zinc release and ERK phosphorylation are required upstream of 12-lipoxygenase activation in peroxynitrite toxicity to mature rat oligodendrocytes. J Biol Chem. 2006; 281:9460-70. PMID: 16431921
37. Zhang Y, Wang H, Li J, Jiminez D A, Levitan E S, Aizenman E, et al. Peroxynitrite induced neuronal apoptosis is mediated by intracellular zinc release and 12-lipoxygenase activation. J Neurosci. 2004; In press.
38. Ho Y, Samarasinghe R, Knoch M E, Lewis M, Aizenman E, DeFranco D B. Selective inhibition of mitogen-activated protein kinase phosphatases by zinc accounts for extracellular signal-regulated kinase 1/2-dependent oxidative neuronal cell death. Molecular pharmacology. 2008; 74:1141-51. PMID: 18635668
39. Sensi S L, Paoletti P, Koh J Y, Aizenman E, Bush A I, Hershfinkel M. The neurophysiology and pathology of brain zinc. J Neurosci. 2011; 31:16076-85. PMID: 22072659
40. Cuajungco M P, Lees G J. Diverse effects of metal chelating agents on the neuronal cytotoxicity of zinc in the hippocampus. Brain research. 1998; 799:97-107.
41. Huang Z, Lippard S J. Illuminating mobile zinc with fluorescence from cuvettes to live cells and tissues. Methods in enzymology. 2012; 505:445-68. PMID: 22289467
42. Kermer P, Klocker N, Bahr M. Long-term effect of inhibition of ced 3-like caspases on the survival of axotomized retinal ganglion cells in vivo. Exp Neurol. 1999; 158:202-5. PMID: 10448432
43. Monnier P P, D'Onofrio P M, Magharious M, Hollander A C, Tassew N, Szydlowska K, et al. Involvement of caspase-6 and caspase-8 in neuronal apoptosis and the regenerative failure of injured retinal ganglion cells. J Neurosci. 2011; 31:10494-505. PMID: 21775595
44. Kwon B K, Liu J, Lam C, Plunet W, Oschipok L W, Hauswirth W, et al. Brain-derived neurotrophic factor gene transfer with adeno-associated viral and lentiviral vectors prevents rubrospinal neuronal atrophy and stimulates regeneration-associated gene expression after acute cervical spinal cord injury. Spine. 2007; 32:1164-73. PMID: 17495772
45. Koeberle P D, Ball A K. Effects of GDNF on retinal ganglion cell survival following axotomy. Vision Res. 1998; 38:1505-15.
46. Akagi T, Kaneda M, Ishii K, Hashikawa T. Differential subcellular localization of zinc in the rat retina. J Histochem Cytochem. 2001; 49:87-96. PMID: 11118481
47. Cole T B, Wenzel H J, Kafer K E, Schwartzkroin P A, Palmiter R D. Elimination of zinc from synaptic vesicles in the intact mouse brain by disruption of the ZnT3 gene. Proceedings of the National Academy of Sciences of the United States of America. 1999; 96:1716-21. PMID: 9990090
48. Wang Z Y, Danscher G, Dahlstrom A, Li J Y. Zinc transporter 3 and zinc ions in the rodent superior cervical ganglion neurons. Neuroscience. 2003; 120:605-16. PMID: 12895501
49. Redenti S, Chappell R L. Localization of zinc transporter-3 (ZnT-3) in mouse retina. Vision research. 2004; 44:3317-21. PMID: 15535999
50. Huang Z, Zhang X A, Bosch M, Smith S J, Lippard S J. Tris(2-pyridylmethyl)amine (TPA) as a membrane-permeable chelator for interception of biological mobile zinc. Metallomics: integrated biometal science. 2013; 5:648-55. PMID: 23715510
51. Meyer R L, Miotke J. Rapid initiation of neurite outgrowth onto laminin from explants of adult mouse retina induced by optic nerve crush. Exp Neurol. 1990; 107:214-21.
52. Danscher G, Stoltenberg M. Zinc-specific autometallographic in vivo selenium methods: tracing of zinc-enriched (ZEN) terminals, ZEN pathways, and pools of zinc ions in a multitude of other ZEN cells. J Histochem Cytochem. 2005; 53:141-53. PMID: 15684327
53. Mook Jo S, Kuk Kim Y, Wang Z, Danscher G. Retrograde tracing of zinc-enriched (ZEN) neuronal somata projecting to the olfactory bulb. Brain Res. 2002; 956:230-5. PMID: 12445690
54. Howell G A, Frederickson C J. A retrograde transport method for mapping zinc-containing fiber systems in the brain. Brain Res. 1990; 515:277-86. PMID: 1694100
55. Wang X, Wang Z Y, Gao H L, Danscher G, Huang L. Localization of ZnT7 and zinc ions in mouse retina—immunohistochemistry and selenium autometallography. Brain Res Bull. 2006; 71:91-6. PMID: 17113933
56. Kim I J, Zhang Y, Meister M, Sanes J R. Laminar restriction of retinal ganglion cell dendrites and axons: subtype-specific developmental patterns revealed with transgenic markers. J Neurosci. 2010; 30:1452-62. PMID: 20107072
57. Kay A R. Evidence for chelatable zinc in the extracellular space of the hippocampus, but little evidence for synaptic release of Zn. J Neurosci. 2003; 23:6847-55. PMID: 12890779
58. Kay A R. Imaging synaptic zinc: promises and perils. Trends in neurosciences. 2006; 29:200-6. PMID: 16515810
59. Nydegger I, Rumschik S M, Kay A R. Zinc is externalized rather than released during synaptic transmission. ACS chemical neuroscience. 2010; 1:728-36. PMID: 21221416
60. Nydegger I, Rumschik S M, Zhao J, Kay A R. Evidence for an extracellular zinc-veneer in rodent brains from experiments with Zn-ionophores and ZnT3 knockouts. ACS chemical neuroscience. 2012; 3:761-6. PMID: 23077720
61. Polazzi E, Mengoni I, Caprini M, Pena-Altamira E, Kurtys E, Monti B. Copper-zinc superoxide dismutase (SOD1) is released by microglial cells and confers neuroprotection against 6-OHDA neurotoxicity. Neuro-Signals. 2013; 21:112-28. PMID: 22572742
62. Maret W. New perspectives of zinc coordination environments in proteins. Journal of inorganic biochemistry. 2012; 111:110-6. PMID: 22196021
63. Maret W. The function of zinc metallothionein: A link between cellular zinc and redox state. Journal of Nutrition. 2000; 130:1455 S-8S.
64. Lee E J, Kim K Y, Gu T H, Moon J I, Kim I B, Lee M Y, et al. Neuronal nitric oxide synthase is expressed in the axotomized ganglion cells of the rat retina. Brain research. 2003; 986:174-80. PMID: 12965242
65. Li J, Ramenaden E R, Peng J, Koito H, Volpe J J, Rosenberg P A. Tumor necrosis factor alpha mediates lipopolysaccharide-induced microglial toxicity to developing oligodendrocytes when astrocytes are present. J Neurosci. 2008; 28:5321-30. PMID: 18480288
66. Kalinchuk A V, Stenberg D, Rosenberg P A, Porkka-Heiskanen T. Inducible and neuronal nitric oxide synthases (NOS) have complementary roles in recovery sleep induction. Eur J Neurosci. 2006; 24:1443-56. PMID: 16987226
67. Kalinchuk A V, Lu Y, Stenberg D, Rosenberg P A, Porkka-Heiskanen T. Nitric oxide production in the basal forebrain is required for recovery sleep. J Neurochem. 2006; 99:483-98. PMID: 17029601
68. Li J, Baud O, Vartanian T, Volpe J J, Rosenberg P A. Peroxynitrite generated by inducible nitric oxide synthase and NADPH oxidase mediates microglial toxicity to oligodendrocytes. Proc Natl Acad Sci USA. 2005; 102:9936-41. PMID: 15998743
69. Zhang Y, Wang H, Li J, Jimenez D A, Levitan E S, Aizenman E, et al. Peroxynitrite-induced neuronal apoptosis is mediated by intracellular zinc release and 12-lipoxygenase activation. J Neurosci. 2004; 24:10616-27. PMID: 15564577
70. Baud O, Li J, Zhang Y, Neve R L, Volpe J J, Rosenberg P A. Nitric oxide-induced cell death in developing oligodendrocytes is associated with mitochondrial dysfunction and apoptosis-inducing factor translocation. The European journal of neuroscience. 2004; 20:1713-26. PMID: 15379992
71. Lu Y, Chung H J, Li Y, Rosenberg P A. NMDA receptor-mediated extracellular adenosine accumulation in rat forebrain neurons in culture is associated with inhibition of adenosine kinase. The European journal of neuroscience. 2003; 17:1213-22. PMID: 12670309
72. Rosenberg P A, Li Y, Le M, Zhang Y. Nitric oxide-stimulated increase in extracellular adenosine accumulation in rat forebrain neurons in culture is associated with ATP hydrolysis and inhibition of adenosine kinase activity. J Neurosci. 2000; 20:6294-301. PMID: 10934281
73. Sindreu C, Palmiter R D, Storm D R. Zinc transporter ZnT-3 regulates presynaptic Erk1/2 signaling and hippocampus-dependent memory. Proc Natl Acad Sci USA. 2011; 108:3366-70. PMID: 21245308
74. Beckman J S, Koppenol W H. Nitric oxide, superoxide, and peroxynitrite: the good, the bad, and ugly. The American journal of physiology. 1996; 271:C1424-37.
75. Beckman J S, Crow J P. Pathological implications of nitric oxide, superoxide and peroxynitrite formation. Biochemical Society transactions. 1993; 21:330-4.
76. Koh J-y, Suh S W, Gwag B J, He Y Y, Hsu C Y, Choi D W. The role of zinc in selective neuronal death after transient global cerebral ischemia. Science (New York, N.Y. 1995; 272:1013-6.
77. Weiss J H, Hartley D M, Koh J-y, Choi D W. AMPA receptor activation potentiates zinc neurotoxicity. Neuron. 1993; 10:43-9.
78. Sheline C T, Ying H S, Ling C S, Canzoniero L M, Choi D W. Depolarization-induced 65zinc influx into cultured cortical neurons. Neurobiology of disease. 2002; 10:41-53. PMID: 12079403
79. Oh H G, Chun Y S, Kim Y, Youn S H, Shin S, Park M K, et al. Modulation of transient receptor potential melastatin related 7 channel by presenilins. Developmental neurobiology. 2012; 72:865-77. PMID: 22102510
80. Inoue K, Branigan D, Xiong Z G. Zinc-induced neurotoxicity mediated by transient receptor potential melastatin 7 channels. J Biol Chem. 2010; 285:7430-9. PMID: 20048154
81. Lichten L A, Cousins R J. Mammalian zinc transporters: nutritional and physiologic regulation. Annual review of nutrition. 2009; 29:153-76. PMID: 19400752
82. Qian J, Xu K, Yoo J, Chen T T, Andrews G, Noebels J L. Knockout of Zn transporters Zip-1 and Zip-3 attenuates seizure-induced CA1 neurodegeneration. J Neurosci. 2011; 31:97-104. PMID: 21209194
83. Leaver S G, Cui Q, Bernard O, Harvey A R. Cooperative effects of bcl-2 and AAV-mediated expression of CNTF on retinal ganglion cell survival and axonal regeneration in adult transgenic mice. Eur J Neurosci. 2006; 24:3323-32. PMID: 17229081
84. Malik J M, Shevtsova Z, Bahr M, Kugler S. Long-term in vivo inhibition of CNS neurodegeneration by Bcl-XL gene transfer. Mol Ther. 2005; 11:373-81. PMID: 15727933
85. Lorber B, Howe M L, Benowitz L I, Irwin N. Mst3b, an Step 20-like kinase, regulates axon regeneration in mature CNS and PNS pathways. Nat Neurosci. 2009; 12:1407-14. PMID: 19855390
86. Fischer D, He Z, Benowitz L I. Counteracting the Nogo receptor enhances optic nerve regeneration if retinal ganglion cells are in an active growth state. J Neurosci. 2004; 24:1646-51. PMID: 14973241
87. Fischer D, Petkova V, Thanos S, Benowitz L I. Switching mature retinal ganglion cells to a robust growth state in vivo: gene expression and synergy with RhoA inactivation. J Neurosci. 2004; 24:8726-40.
88. Moore D L, Blackmore M G, Hu Y, Kaestner K H, Bixby J L, Lemmon V P, et al. KLF family members regulate intrinsic axon regeneration ability. Science. 2009; 326:298-301. PMID: 19815778
89. Goldberg J L, Klassen M P, Hua Y, Barres B A. Amacrine-signaled loss of intrinsic axon growth ability by retinal ganglion cells. Science. 2002; 296:1860-4.
90. Yin Y, Cui Q, Li Y, Irwin N, Fischer D, Harvey A R, et al. Macrophage-derived factors stimulate optic nerve regeneration. J Neurosci. 2003; 23:2284-93.
91. Kay A R. Detecting and minimizing zinc contamination in physiological solutions. BMC physiology. 2004; 4:4. PMID: 15113426
92. Tedeschi A, Bradke F. The DLK signalling pathway-a double-edged sword in neural development and regeneration. EMBO reports. 2013. PMID: 23681442
93. Hassig C A, Tong J K, Fleischer T C, Owa T, Grable P G, Ayer D E, et al. A role for histone deacetylase activity in HDAC1-mediated transcriptional repression. Proc Natl Acad Sci USA. 1998; 95:3519-24. PMID: 9520398
94. Gaub P, Joshi Y, Wuttke A, Naumann U, Schnichels S, Heiduschka P, et al. The histone acetyltransferase p300 promotes intrinsic axonal regeneration. Brain. 2011; 134:2134-48. PMID: 21705428
95. Gaub P, Tedeschi A, Puttagunta R, Nguyen T, Schmandke A, Di Giovanni S HDAC inhibition promotes neuronal outgrowth and counteracts growth cone collapse through CBP/p300 and P/CAF-dependent p53 acetylation. Cell Death Differ. 2010; 17:1392-408. PMID: 20094059
96. Koriyama Y, Takagi Y, Chiba K, Yamazaki M, Arai K, Matsukawa T, et al. Neuritogenic activity of a genipin derivative in retinal ganglion cells is mediated by retinoic acid receptor beta expression through nitric oxide/S-nitrosylation signaling. J Neurochem. 2011; 119:1232-42. PMID: 21995424
97. Irwin N, Li Y M, O'Toole J E, Benowitz L I. Mst3b, a purine-sensitive Step 20-like protein kinase, regulates axon outgrowth. Proc Natl Acad Sci USA. 2006; 103:18320-5. PMID: 17114295
98. Lu T J, Huang C Y, Yuan C J, Lee Y C, Leu T H, Chang W C, et al. Zinc ion acts as a cofactor for serine/threonine kinase MST3 and has a distinct role in autophosphorylation of MST3. Journal of inorganic biochemistry. 2005; 99:1306-13. PMID: 15917084
99. Besser L, Chorin E, Sekler I, Silverman W F, Atkin S, Russell J T, et al. Synaptically released zinc triggers metabotropic signaling via a zinc-sensing receptor in the hippocampus. J Neurosci. 2009; 29:2890-901. PMID: 19261885
100. Yu S P, Yeh C H, Sensi S L, Gwag B J, Canzoniero L M, Farhangrazi Z S, et al. Mediation of neuronal apoptosis by enhancement of outward potassium current. Science (New York, N.Y. 1997; 278:114-7.
101. Redman P T, Hartnett K A, Aras M A, Levitan E S, Aizenman E. Regulation of apoptotic potassium currents by coordinated zinc-dependent signalling. J Physiol. 2009; 587:4393-404. PMID: 19622611
102. Koeberle P D, Wang Y, Schlichter L C. Kv1.1 and Kv1.3 channels contribute to the degeneration of retinal ganglion cells after optic nerve transection in vivo. Cell Death Differ. 2010; 17:134-44. PMID: 19696788
103. Degterev A, Yuan J. Expansion and evolution of cell death programmes. Nat Rev Mol Cell Biol. 2008; 9:378-90. PMID: 18414491
104. Hardwick J M, Chen Y B, Jonas E A. Multipolar functions of BCL-2 proteins link energetics to apoptosis. Trends in cell biology. 2012; 22:318-28. PMID: 22560661
105. Kermer P, Klocker N, Labes M, Bahr M Inhibition of CPP32-like proteases rescues axotomized retinal ganglion cells from secondary cell death in vivo. J Neurosci. 1998; 18:4656-62.
106. Fischer D, Petkova V, Thanos S, Benowitz L I. Switching mature retinal ganglion cells to a robust growth state in vivo: gene expression and synergy with RhoA inactivation. J Neurosci. 2004; 24:8726-40. PMID: 15470139
107. Hille B. Ion Channels of Excitable Membranes. 3rd ed. Sunderland, M A: Sinauer; 2001.
108. Grissmer S, Nguyen A N, Aiyar J, Hanson D C, Mather R J, Gutman G A, et al. Pharmacological characterization of five cloned voltage-gated K+ channels, types Kv1.1, 1.2, 1.3, 1.5, and 3.1, stably expressed in mammalian cell lines. Molecular pharmacology. 1994; 45:1227-34. PMID: 7517498
109. Rudy B, McBain C J. Kv3 channels: voltage-gated K+ channels designed for high-frequency repetitive firing. Trends in neurosciences. 2001; 24:517-26. PMID: 11506885
110. Wang H S, Pan Z, Shi W, Brown B S, Wymore R S, Cohen I S, et al. KCNQ2 and KCNQ3 potassium channel subunits: molecular correlates of the M-channel. Science (New York, N.Y. 1998; 282:1890-3. PMID: 9836639
111. Pal S, Hartnett K A, Nerbonne J M, Levitan E S, Aizenman E. Mediation of neuronal apoptosis by Kv2.1-encoded potassium channels. J Neurosci. 2003; 23:4798-802. PMID: 12832499
112. Hughes F M, Jr., Cidlowski J A. Potassium is a critical regulator of apoptotic enzymes in vitro and in vivo. Advances in enzyme regulation. 1999; 39:157-71. PMID: 10470372
113. Ofengeim D, Chen Y B, Miyawaki T, Li H, Sacchetti S, Flannery R J, et al. N-terminally cleaved Bcl-xL mediates ischemia-induced neuronal death. Nat Neurosci. 2012; 15:574-80. PMID: 22366758
114. Yeung S Y, Thompson D, Wang Z, Fedida D, Robertson B. Modulation of Kv3 subfamily potassium currents by the sea anemone toxin BDS: significance for CNS and biophysical studies. J Neurosci. 2005; 25:8735-45. PMID: 16177043
115. Diochot S, Schweitz H, Beress L, Lazdunski M. Sea anemone peptides with a specific blocking activity against the fast inactivating potassium channel Kv3.4. J Biol Chem. 1998; 273:6744-9. PMID: 9506974
116. Lingor P, Koeberle P, Kugler S, Bahr M. Down-regulation of apoptosis mediators by RNAi inhibits axotomy-induced retinal ganglion cell death in vivo. Brain. 2005; 128:550-8. PMID: 15659426
117. Koeberle P D, Schlichter L C. Targeting K(V) channels rescues retinal ganglion cells in vivo directly and by reducing inflammation. Channels (Austin). 2010; 4:337-46. PMID: 20699649
118. Clem R J, Cheng E H, Karp C L, Kirsch D G, Ueno K, Takahashi A, et al. Modulation of cell death by Bcl-XL through caspase interaction. Proceedings of the National Academy of Sciences of the United States of America. 1998; 95:554-9. PMID: 9435230
119. Kuznetsov K I, Grygorov O O, Maslov V Y, Veselovsky N S, Fedulova S A. Kv3 channels modulate calcium signals induced by fast firing patterns in the rat retinal ganglion cells. Cell calcium. 2012; 52:405-11. PMID: 22831914
120. Henne J, Pottering S, Jeserich G. Voltage-gated potassium channels in retinal ganglion cells of trout: a combined biophysical, pharmacological, and single-cell RT-PCR approach. Journal of neuroscience research. 2000; 62:629-37. PMID: 11104501
121. Zhang X, Yang D, Hughes B A. KCNQ5/K(v)7.5 potassium channel expression and subcellular localization in primate retinal pigment epithelium and neural retina. Am J Physiol Cell Physiol. 2011; 301:C1017-26. PMID: 21795522
122. Diem R, Meyer R, Weishaupt J H, Bahr M. Reduction of potassium currents and phosphatidylinositol 3-kinase-dependent AKT phosphorylation by tumor necrosis factor-(alpha) rescues axotomized retinal ganglion cells from retrograde cell death in vivo. J Neurosci. 2001; 21:2058-66. PMID: 11245689
123. Aras M A, Aizenman E. Redox regulation of intracellular zinc: molecular signaling in the life and death of neurons. Antioxid Redox Signal. 2011; 15:2249-63. PMID: 20849376
124. Radford R J, Lippard S J. Chelators for investigating zinc metalloneurochemistry. Curr Opin Chem Biol. 2013; 17:129-36. PMID: 23478014
125. Kabakov A Y, Rosenberg P A. Evidence for change in current-flux coupling of GLT1 at high glutamate concentrations in rat primary forebrain neurons and GLT1a-expressing COS-7 cells. The European journal of neuroscience. 2009; 30:186-95. PMID: 19614985
126. Petr G T, Bakradze E, Frederick N M, Wang J, Armsen W, Aizenman E, et al. Glutamate transporter expression and function in a striatal neuronal model of Huntington's disease. Neurochemistry international. 2013; 62:973-81. PMID: 23507328
127. Imredy J P, Chen C, MacKinnon R. A snake toxin inhibitor of inward rectifier potassium channel ROMK1. Biochemistry. 1998; 37:14867-74. PMID: 9778362
128. Akamine T, Nishimura Y, Ito K, Uji Y, Yamamoto T. Effects of haloperidol on K(+) currents in acutely isolated rat retinal ganglion cells. Invest Ophthalmol V is Sci. 2002; 43:1257-61. PMID: 11923273
129. Ishida A T. Ion channel components of retinal ganglion cells. Progress in retinal and eye research. 1995; 15:261-80.
130. Weick M, Demb J B. Delayed-rectifier K channels contribute to contrast adaptation in mammalian retinal ganglion cells. Neuron. 2011; 71:166-79. PMID: 21745646
131. Lilley S, Robbins J. The rat retinal ganglion cell in culture: an accessible CNS neurone. Journal of pharmacological and toxicological methods. 2005; 51:209-20. PMID: 15862466
132. Ozaita A, Petit-Jacques J, Volgyi B, Ho C S, Joho R H, Bloomfield S A, et al. A unique role for Kv3 voltage-gated potassium channels in starburst amacrine cell signaling in mouse retina. J Neurosci. 2004; 24:7335-43. PMID: 15317859
133. Koizumi A, Jakobs T C, Masland R H. Inward rectifying currents stabilize the membrane potential in dendrites of mouse amacrine cells: patch-clamp recordings and single-cell RT-PCR. Mol Vis. 2004; 10:328-40. PMID: 15152185
134. Henne J, Jeserich G. Maturation of spiking activity in trout retinal ganglion cells coincides with upregulation of Kv3.1- and BK-related potassium channels. Journal of neuroscience research. 2004; 75:44-54. PMID: 14689447
135. Chen L, Yu Y C, Zhao J W, Yang X L. Inwardly rectifying potassium channels in rat retinal ganglion cells. The European journal of neuroscience. 2004; 20:956-64. PMID: 15305864
136. Shin J E, Cho Y, Beirowski B, Milbrandt J, Cavalli V, DiAntonio A. Dual leucine zipper kinase is required for retrograde injury signaling and axonal regeneration. Neuron. 2012; 74:1015-22. PMID: 22726832.

What is claimed:

1. A method of promoting axonal outgrowth of a neuron comprising contacting the neuron with an effective amount of a chelating agent, to thereby promote axonal outgrowth in the neuron.

2. The method of claim 1, wherein the neuron is an injured neuron.

3. The method of claim 2, wherein the injured neuron results from acute traumatic injury.

4. The method of claim 1, wherein the neuron is further contacted with one or more additional agents that promote axonal outgrowth.

5. The method of claim 4, wherein the agent that promotes axonal outgrowth is selected from the group consisting of inosine, oncomodulin, a pten inhibitor, and combinations thereof.

6. The method of claim 1, wherein the chelating agent binds zinc.

7. The method of claim 1, wherein the chelating agent binds divalent cations intracellularly, extracellularly, or both intracellularly and extracellularly.

8. The method of claim 1, wherein the chelating agent is selected from the group consisting of TPEN, ZX1, TPA, ZX1E, EDTA saturated with Ca²⁺, and combinations thereof.

9. A method of treating a subject for a CNS lesion, comprising, administering to the subject a therapeutically effective amount of a chelating agent, wherein administering results in contacting one or more lesioned CNS neurons of the subject with the chelating agent, to thereby promote regeneration in the CNS neurons.

10. The method of claim 9, wherein the chelating agent is selected from the group consisting of TPEN, ZX1, TPA, ZX1E, EDTA saturated with Ca²⁺, and combinations thereof.

11. The method of claim 9, wherein the CNS lesion results from an acute traumatic injury.

12. The method of claim 9 wherein administration first occurs following an injury that results in the lesion.

13. The method of claim 9, wherein the CNS lesion results from an acute traumatic injury.

14. The method of claim 13, wherein the acute traumatic injury is selected from the group consisting of stroke, acute spinal cord injury, and traumatic brain injury.

15. The method of claim 9, wherein the lesioned CNS neuron is in the optic nerve.

16. The method of claim 9, wherein the lesioned CNS neuron is in the spinal cord of a patient, and the inhibitor is intrathecally administered to the patient.

17. The method of claim 9, wherein lesioned CNS neuron is a sensory neuron.

18. The method of claim 9, wherein the chelating agent is administered locally at a site of axonal injury, or at a site of origin of an injured neuron.

19. The method of claim 9, wherein one or more additional agents that promote axonal outgrowth are administered to the subject.

20. A device for promoting regeneration in a lesioned central nervous system (CNS) neuron, comprising a reservoir loaded with a premeasured and contained amount of a therapeutically effective amount of a chelating agent, and specifically adapted for implementing the method of claim 9.
